(19) United States
(12) Patent Application Publication
(10) Pub. No.: US 2022/0409708 A1
(43) Pub. Date: Dec. 29, 2022

(54) SMALL LIPID NANOPARTICLES, AND CANCER VACCINE INCLUDING SAME

(71) Applicant: KOREA ADVANCED INSTITUTE OF SCIENCE AND TECHNOLOGY, Daejeon (KR)

(72) Inventors: Sang Yong JON, Daejeon (KR); Yu Jin KIM, Jeollanam-do (KR); Suk Mo KANG, Daejeon (KR)

(21) Appl. No.: 17/773,658

(22) PCT Filed: Nov. 1, 2019
(86) PCT No.: PCT/KR2019/014741

§ 371 Date: May 2, 2022

(51) Int. Cl. A61K 39/00; A61K 39/395; A61P 35/00; A61K 39/39

(52) U.S. Cl. CPC A61K 39/0012; A61K 39/0011; A61K 39/3955; A61P 35/00; A61K 39/39; A61K 2039/55561

(57) ABSTRACT

The present invention relates to small lipid nanoparticles, a small lipid nanoparticle (SLNP)-based nanovaccine platform including same, and a combination treatment regimen with an immune checkpoint inhibitor. Lipid nanoparticies according to the present invention can easily deliver antigens and anionic drugs into cells, and exhibit strong anti-tumor effects when loaded with tumor-associated antigens. Particularly, a cancer vaccine kit according to the present invention including lipid nanoparticles according to the present invention as a first vaccine composition and lipid nanoparticles and an immune checkpoint inhibitor as a second vaccine composition can be used to effectively suppress tumor regrowth and recurrence triggered by the occurrence of immunosuppression against a cancer nanovaccine.

### TECHNICAL FIELD

The present invention has been made by project number 2018R1A3B1052661 under the support of the Ministry of Science and ICT of the Republic of Korea, the research management institution for the above project is the National Research Foundation of Korea, the research project name is “Basic Research Project in Science and Engineering Field”, the research task name is “Tumor Microenvironment Target and Sensitive Precision Bio-Nanomedicine Research Group”, the responsible authority is the Korea Advanced Institute of Science and Technology, and the research period is 2019 Mar. 1˜2020 Feb. 29.

Also, the present invention has been made by project number 2018M3A9B5023527 under the support of the Ministry of Science and ICT of the Republic of Korea, the research management institution for the above project is the National Research Foundation of Korea, the research project name is “Original Technology Development Project”, the research task name is “Development of tumor microenvironment target and sensitive drug delivery platform technology”, and the responsible authority is the Korea Advanced Institute of Science and Technology, and the research period is 2019 Jan. 1˜2019 Dec. 31.

The present invention relates to small lipid nanoparticles, a small lipid nanoparticle (SLNP)-based nanovaccine platform including same, and a combination treatment regimen with an immune checkpoint inhibitor.

### BACKGROUND ART

Cancer nanovaccines based on nanomaterials that carry tumor-associated antigens or tumor-specific neoantigens have shown promising therapeutic efficacy in preclinical animal models, but the clinical use of these nanovaccines has been limited. Despite the ability of cancer nanovaccines to expand the pool of tumor-specific T cells, the occurrence of immune evasion and immunosuppression in the tumor microenvironment (TME) is considered to cause poor therapeutic response in nanovaccine trials. Particularly, the increased expression at the immune checkpoint of programmed death ligand 1 (PD-L1), which binds to the programmed cell death-1 receptor (PD-1) on T cells, has been reported to limit the therapeutic efficacy of these vaccines by inducing adaptive resistance of tumors to vaccine-mediated immune responses. This immunosuppression could be overcome by a combination of immune checkpoint blockers (ICBs) such as anti-PD-1 and anti-PD-L1 antibodies and cancer nanovaccines, thereby enhancing the therapeutic effect. However, to date, there are few studies that have evaluated the optimal timing and sequence of cancer nanovaccines and ICBs. For example, it is unclear whether concurrent treatment leads to better treatment outcomes or whether sequential treatment leads to better treatment outcomes. Moreover, the optimal combination timing of the two treatment modalities has not been determined. A rational approach is needed to determine the timing and order of administration of cancer nanovaccines and ICB. The present invention relates to a novel combination treatment regimen that can improve nanovaccine-induced adaptive immune resistance and thus effectively suppress tumor growth and recurrence. Further, the present invention has produced a neutral-charged small lipid nanoparticles (SLNPs) that function as both an antigen and an adjuvant-carrying nanovaccine in order to investigate whether rationally designed antigen-carrying nanomaterials are suitable candidates for cancer nanovaccines. The present inventors has found that sequential administration and combination of nanovaccine and anti-PD1 antibody can show effective anti-tumor effect and inhibition of tumor recurrence, and completed the present invention.

### PRIOR ART LITERATURE

### Non-Patent Literature

Shirota et al, Vaccines 2015, 3, 390-407.

### SUMMARY

### Technical Problem

It is an object of the present invention to provide a lipid nanoparticle comprising an antigen, a phospholipid, a cationic lipid, and an adjuvant.

It is another object of the present invention to provide a vaccine composition comprising the above-mentioned lipid nanoparticle as an active ingredient.

It is yet another object of the present invention to provide a cancer vaccine kit which comprises a lipid nanoparticle including a tumor-associated antigen, a phospholipid, a cationic lipid, and comprises an anionic drug as a first vaccine composition and the lipid nanoparticles and an immune checkpoint inhibitor as a second vaccine composition.

### Technical Solution

According to an embodiment of the present invention, there is provided a lipid nanoparticle comprising an antigen, a phospholipid, a cationic lipid, and an adjuvant.

In one embodiment of the present invention, the antigen is a tumor-associated antigen.

More specifically, the tumor-associated antigen is selected from the group consisting of MAGE-1, MAGE-2, MAGE-3, MAGE-12, BAGE, GAGE, NY-ESO-1, tyrosinase, TRP-1, TRP-2, gp100, MART-1, MCIR, Ig idotype, CDK4, Caspase-B, beta-catenin, CLA, BCR/ABL, mutated p21/ras, mutated p53, proteinase 3, WT1, MUC-1, Her2/neu, PAP, PSA, PSMA, G250, HPV E6/E7, EBV LMP2a, CEA, alpha-Fetoprotein, 5T4, onco-trophoblast glycoprotein, and the like, but is not limited thereto. Those skilled in the art will readily appreciate that various antigens applicable to cancer vaccines in the art can be applied.

In one embodiment of the present invention, the tumor (or cancer) includes breast cancer, head and neck cancer, bladder cancer, stomach cancer, rectal/colon cancer, pancreatic cancer, lung cancer, melanoma, prostate cancer, kidney cancer, liver cancer, cervical cancer, and the like, but is not limited thereto.

In one embodiment of the present invention, the phospholipid is a phospholipid having 14 to 22 aliphatic carbon atoms.

In a specific embodiment of the present invention, the phospholipid is at least one phospholipid selected from the group consisting of DSPE-PEG derivative including 1,2-Distearoyl-sn-glycero-3-phosphoethanolamine (DSPE), 1,2-Distearoyl-sn-glycero-3-phosphoethanolamine-N-[carboxy(polyethylene glycol)-1000](DSP E-P EG₁₀₀₀), functionalized DSPE-PEG derivative including 1,2-Distearoyl-sn-glycero-3-phosphoethanolamine-N-[PDP(polyethylene glycol)-2000](DSPE-PEG₂₀₀₀-PDP), 1,2-Distearoyl-sn-glycero-3-phosphoethanolamine-N-[maleimide(polyethylene glycol)-2000](DSPE-PEG₂₀₀₀-Maleimide) and the like, fluorescence-labeled phospholipid including 1,2-Dioleoyl-sn-glycero-3-phosphoethanolamine (DOPE) and 1,2-Dipalmitoyl-sn-glycero-3-phosphoethanolamine-N-(lissamine rhodamine B sulfonyl) (DPP E-Rhodam ine), 1,2-Didecanoyl-sn-glycero-3-phosphocholine (DDPC), 1,2-Dierucoyl-sn-glycero-3-phosphate (DEPA), 1,2-Dierucoyl-sn-glycero-3-phosphocholine (DEPC), 1,2-Dierucoyl-sn-glycero-3-phosphoethanolamine (DEPE), 2-Dierucoyl-sn-glycero-3-Phospho-rac-(1-glycerol) (DEPG), 1,2-Dilinoleoyl-sn-glycero-3-phosphocholine (DLOPC), 1,2-Dilauroyl-sn-glycero-3-phosphate (DLPA), 1,2-Dilauroyl-sn-glycero-3-phosphocholine (DLPE), 1,2-Dilauroyl-sn-glycero-3-Phospho-rac-(1-glycerol) (DLPG), 1,2-Dilauroyl-sn-glycero-3-phosphoserine (DLPS), 1,2-Dimyristoyl-sn-glycero-3-phosphate (DMPA), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3-Phospho-rac-(1-glycerol) (DMPG), 1,2-Dimyristoyl-sn-glycero-3-phosphoserine (DMPS), 1,2-Dioleoyl-sn-glycero-3-phosphate (DOPA), 1,2-Dioleoyl-sn-glycero-3-phosphocholine (DOPC), 1,2-Dioleoyl-sn-glycero-3-Phospho-rac-(1-glycerol) (DOPG), 1, 2-Dioleoyl-sn-glycero-3-phosphoserine (DOPS), 1,2-Dipalmitoyl-sn-glycero-3-phosphate (DPPA), 1,2-Dipalmitoyl-sn-glycero-3-phosphocholine(DPPC), 1,2-Dipalmitoyl-sn-glycero-3-phosphoethanolamine (DPPE), 1,2-Dipalmitoyl-sn-glycero-3-Phospho-rac-(1-glycerol) (DPPG), 1,2-Dipalmitoyl-sn-glycero-3-phosphoserine(DPPS), 1,2-Distearoyl-sn-glycero-3-phosphate(DSPA), 1,2-D istearoyl-sn-glycero-3-phosphocholine(DSPC), 1,2-Distearoyl-sn-glycero-3-Phospho-rac-(1-glycerol) (DSPG), 1,2-Distearoyl-sn-glycero-3-phosphoserine (DSPS), Egg-PC (EPC), Hydrogenated Egg PC (HEPC), Hydrogenated Soy PC(HSPC), 1-Myristoyl-sn-glycero-3-phosphocholine (LYSOPC MYRISTIC), 1-Palmitoyl-sn-glycero-3-phosphocholine (LYSOPC PALM ITIC), 1-Stearoyl-sn-glycero-3-phosphocholine(LYSOPC STEARIC), 1-Myristoyl-2-palmitoyl-sn-glycero 3-phosphocholine (Milk Sphingomyelin MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2-oleoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3-Phospho-rac-(1-glycerol) (POPG), 1-Palmitoyl-2-stearoyl-sn-glycero-3-phosphocholine (PSPC), 1-Stearoyl-2-myristoyl-sn-glycero-3-phosphocholine(SMPC), 1-Stearoyl-2-oleoyl-sn-glycero-3-phosphocholine (SOPC) and 1-Stearoyl-2-palmitoyl-sn-glycero-3-phosphocholine (SPPC), but is not limited thereto.

In one embodiment of the present invention, the cationic lipid is at least one cationic lipid selected from the group consisting of O-alkyl phosphatidylcholine derivatives including Dimethyldioctadecyl-ammoniumbromide (DDAB), Dimethyldioctadecylammonium (DDAB), (N,N-dimethyl-N-([2-sperminecarboxamido]ethyl)-2,3-bis(dioleyloxy)-1-propaniminium pentahydrochloride) (DOSPA), (N-[1-(2,3-dioleyloxy)propyl]-N,N,N-trimethylammonium) (DOTMA), (N-[1-(2,3-dioleoyloxy)propyl]-N,N,N-trimethylammonium) (DOTAP), 3β-[N-(N′,N′-dimethylaminoethane)-carbamoyl]cholesterol (DC-Chol), N4-Cholesteryl-Spermine (GL67), 1,2-dioleyloxy-3-dimethylaminopropane (DODMA), 1,2-dilauroyl-sn-glycero-3-ethylphosphocholine (12:0 EPC), and DAP derivatives including N-(4-carboxybenzyl)-N,N-dimethyl-2,3-bis(oleoyloxy)propan-1-aminium (DOBAQ) and 1,2-distearoyl-3-dimethylammonium-propane (18:0 DAP), but is not limited thereto.

In one embodiment of the present invention, the cationic lipid is a cationic cholesterol derivative. Specifically, the cationic cholesterol derivative is Monoarginine-cholesterol (MA-Chol).

In one embodiment of the present invention, the lipid nanoparticle may include an anionic drug in addition to the adjuvant. In a specific embodiment of the present invention, the anionic drug is an oligonucleotide, an aptamer, mRNA, siRNA, miRNA, ora combination thereof.

In one embodiment of the present invention, the adjuvant is an immunostimulatory single-or double-stranded oligonucleotide, an immunostimulatory small-molecule compound, or a combination thereof.

In one embodiment of the present invention, the immunostimulatory single- or double-stranded oligonucleotide are known as a useful adjuvant (auxiliary immune agent).

They often contain a CpG motif (a dinucleotide sequence containing unmethylated cytosine linked to guanosine). Oligonucleotides comprising a TpG motif, a palindrome arrangement, a plurality of contiguous thymidine nucleotides (e.g., TTTT), a plurality of contiguous cytosine nucleotides (e.g., CCCC) or a poly(dG) arrangement are also a known adjuvant like double-stranded RNA. Any of these various immunostimulatory oligonucleotides can be used in conjunction with the present invention without limitation.

The oligonucleotide typically has 10˜100 nucleotides, for example 15˜50 nucleotides, 20˜30 nucleotides, or 25˜28 nucleotides. It is typically a single-stranded.

The oligonucleotide may include only natural nucleotides, only non-natural nucleotides, or a mixture of both. For example, the oligonucleotide may contain one or more phosphorothioate bonds, and/or may be one or more 2′-O-methyl modifications.

In a specific embodiment of the present invention, the single- or double-stranded oligonucleotide is a CpG oligonucleotide, a STING-active oligonucleotide, or a combination thereof.

As used herein, the term “Stimulator of Interferon Genes (STING)” means a stimulator of interferon genes (STING), which is a molecule that plays a major role in the innate immune response. STING comprises five putative transmembrane (TM) regions, premodminantly resides in the endoplasmic reticulum (ER), and is able to activate both NF-kB and IRF3 transcription pathways to induce type I IFG and exert a potent anti-viral status following expression (see U.S. patent application Ser. Nos. 13/057,662 and PCT/US2009/052767). Loss of STING rendered murine embryonic fibroblasts (−/−MEFs) that reduces the ability of polyIC to activate type I IFN, lacks STING produced by targeted homologous recombination, and is susceptible to vesicular stomatitis virus (VSV) infection. In the absence of STING, the DNA-mediated type I IFN response is inhibited, indicating that STING can play an important role in recognizing DNA from viruses, bacteria and other pathogens that can infect cells. Yeast double hybridization and co-immunoprecipitation studies have showed that STING interacts with RIG-I and Ssr2/TRAPβ (member of the transloconassociated protein (TRAP) complex required for protein transport across the ER membrane after translation). RNAi removal of TRAPβ inhibited STING function and prevented the production of type I IFN in response to polyIC. Further experiments have shown that STING itself binds to nucleic acids including single- and double-stranded DNA, for example from pathogens or apoptotic DNA, and plays a central role in regulating proinflammatory gene expression in DNA-mediated arthritis and inflammatory conditions such as cancer. Various novel methods for up-regulating STING expression or function, and various novel compositions for up-regulating STING expression or function are described herein along with further characterization of other cellular molecules that interact with STING. These findings allow the design of new adjuvants, vaccines and therapies to modulate the immune system and other systems.

The STING-active oligonucleotide may be a nucleic acid molecule that binds the STING function to STING. The STING-binding nucleic acid molecule may be a single-stranded DNA of 40 to 150 base pairs in length or a double-stranded DNA of at least 40 to 150, 60 to 120, 80 to 100, or 85 to 95 base pairs in length. The STING-binding nucleic acid molecule can be, for example, nuclease resistant made from nuclease resistant nucleotides. STING-binding nucleic acid molecule can also bind to a molecule that facilitates transmembrane transport. In such methods, the disease or disorder may be a DNA-dependent inflammatory disease. Also described herein are methods of treating cancer in a subject having a cancerous tumor infiltrated with inflammatory immune cells. Such methods may include administering to the subject any amount of a pharmaceutical composition comprising an agent that down-regulates the function or expression of STING and a pharmaceutically acceptable carrier.

More specifically, the oligonucleotide is an antisense oligonucleotide, a CpG oligonucleotide, or a combination thereof.

As used herein, CpG oligonucleotide or CpG oligodeoxynucleotide (CpG ODN) is a short single-stranded synthetic DNA molecule comprising an unmethylated cytosine triphosphate deoxynucleotide (“C”) and a guanine triphosphate deoxynucleotide (“G”), which is known as an immunostimulant. When included as a component of the nanovaccine of the present invention, the CpG serves as an adjuvant that enhances the immune response of dendritic cells.

The immunostimulatory small-molecule compound is also called a small molecule adjuvant, and includes a synthetic small-molecule adjuvant and a natural small-molecule adjuvant. Examples of the immunostimulatory small-molecule compound or small-molecule adjuvant include monophosphoryl lipid A, Muramyl dipeptide, Bryostatin-1, Mannide monooleate (Montanide ISA 720), Squalene, QS21, Bis-(3′,5′)-cyclic dimeric guanosine monophosphate, PAM2CSK4, PAM3CSK4, Imiquimod, Resiquimod, Gardiquimod, c1075, c1097, Levamisole, 48/80, Bupivacaine, Isatoribine, Bestatin, Sm360320, Loxoribine and the like, but are not limited thereto. Small molecule adjuvants are described in Flower DR et al. (Expert Opin Drug Discov. 2012 September; 7(9):807-17.).

In another aspect of the present invention, there is provided a vaccine composition comprising the above-mentioned lipid nanoparticles as an active ingredient.

The vaccine composition is a pharmaceutical composition, and includes a pharmaceutically acceptable excipient, or carrier, in addition to the above-mentioned lipid nanoparticles.

As used herein, the term “pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

The subject to which the present vaccine composition is applied can be any animal, and specifically is a mammal such as a human, mouse, rat, hamster, guinea pig, rabbit, cat, dog, monkey, cow, horse, pig, and the like. Humans are most preferred.

The vaccine compositions can be formulated as freeze-dried or liquid preparations according to any means suitable in the art. Non-limiting examples of liquid form preparations include solutions, suspensions, syrups, slurries, and emulsions. Suitable liquid carriers include any suitable organic or inorganic solvent, for example, water, alcohol, saline solution, buffered saline solution, physiological saline solution, dextrose solution, water propylene glycol solutions, and the like, preferably in sterile form.

The vaccine compositions can be formulated in either neutral or salt forms. Pharmaceutically acceptable salts include the acid addition salts (formed with the free amino groups of the active polypeptides) and which are formed with inorganic acids such as, for example, hydrochloric or phosphoric acids, or organic acids such as acetic, oxalic, tartaric, mandelic, and the like. Salts formed from free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccine compositions are preferably formulated for inoculation or injection into the subject. For injection, the vaccine compositions of the invention can be formulated in aqueous solutions such as water or alcohol, or in physiologically compatible buffers such as Hanks's solution, Ringer's solution, or physiological saline buffer. The solution can contain formulatory agents such as suspending, preserving, stabilizing and/or dispersing agents. Injection formulations can also be prepared as solid form preparations which are intended to be converted, shortly before use, to liquid form preparations suitable for injection, for example, by constitution with a suitable vehicle, such as sterile water, saline solution, or alcohol, before use.

The vaccine compositions can also be formulated in sustained release vehicles or depot preparations. Such long acting formulations can be administered by inoculation or implantation (for example subcutaneously or intramuscularly) or by injection. Thus, for example, the vaccine compositions can be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well-known examples of delivery vehicles suitable for use as carriers.

The vaccine compositions can comprise agents that enhance the protective efficacy of the vaccine, such as adjuvants. Adjuvants include any compound or compounds that act to increase a protective immune response to the peptide antigen, thereby reducing the quantity of antigen necessary in the vaccine, and/or the frequency of administration necessary to generate a protective immune response. Adjuvants can include for example, emulsifiers, muramyl dipeptides, pyridine, aqueous adjuvants such as aluminum hydroxide, chitosan-based adjuvants, and any of the various saponins, oils, and other substances known in the art, such as Amphigen, LPS, bacterial cell wall extracts, bacterial DNA, CpG sequences, synthetic oligonucleotides and combinations thereof (see Schijns et al. (2000) Curr. Opin. Immunol. 12:456), Mycobacterialphlei (M. phlei) cell wall extract (MCWE) (U.S. Pat. No. 4,744,984), M. phlei DNA (M-DNA), and M-DNA-M. phlei cell wall complex (MCC). Compounds which can be used as emulsifiers include natural and synthetic emulsifying agents, as well as anionic, cationic and nonionic compounds. Among the synthetic compounds, anionic emulsifying agents include, for example, the calcium, sodium and ammonium salts of lauric and oleic acid, the calcium, magnesium and aluminum salts of fatty acids, and organic sulfonates such as sodium lauryl sulfate. Synthetic cationic agents include, for example, cetyltrhethylammonlum bromide, while synthetic nonionic agents are exemplified by glycerylesters (e.g., glyceryl monostearate), polyoxyethylene glycol esters and ethers, and the sorbitan fatty acid esters (e.g., sorbitan monopalmitate) and their polyoxyethylene derivatives (e.g., polyoxyethylene sorbitan monopalmitate). Natural emulsifying agents include acacia, gelatin, lecithin and cholesterol.

Other suitable adjuvants can be formed with an oil component, such as a single oil, a mixture of oils, a water-in-oil emulsion, or an oil-in-water emulsion. The oil can be a mineral oil, a vegetable oil, or an animal oil. Mineral oils are liquid hydrocarbons obtained from petrolatum via a distillation technique, and are also referred to in the art as liquid paraffin, liquid petrolatum, or white mineral oil. Suitable animal oils include, for example, cod liver oil, halibut oil, menhaden oil, orange roughy oil and shark liver oil, all of which are available commercially. Suitable vegetable oils, include, for example, canola oil, almond oil, cottonseed oil, corn oil, olive oil, peanut oil, safflower oil, sesame oil, soybean oil, and the like. Freund's Complete Adjuvant (FCA) and Freund's Incomplete Adjuvant (FIA) are two common adjuvants that are commonly used in vaccine preparations, and are also suitable for use in the present invention. Both FCA and FIA are water-in-mineral oil emulsions; however, FCA also contains a killed Mycobacterium sp.

Immunomodulatory cytokines can also be used in the vaccine compositions to enhance vaccine efficacy, for example, as an adjuvant. Non-limiting examples of such cytokines include interferon alpha (IFN-α), interleukin-2 (IL-2), and granulocyte macrophage-colony stimulating factor (GM-CSF), or combinations thereof. GM-CSF is highly preferred.

Vaccine compositions comprising antigens and further comprising adjuvants can be prepared using techniques well known to those skilled in the art including, but not limited to, mixing, sonication and microfluidation. The adjuvant can comprise from about 10% to about 50% (v/v) of the vaccine composition, more preferably about 20% to about 40% (v/v), and more preferably about 20% to about 30% (v/v), or any integer within these ranges. About 25% (v/v) is highly preferred.

The vaccine compositions can be administrated by infusion or injection (e.g., intravenously, intramuscularly, intracutaneously, subcutaneously, intrathecal, intraduodenally, intraperitoneally, and the like). The vaccine compositions can also be administered intranasally, vaginally, rectally, orally, or transdermally. Additionally, vaccine compositions can be administered by “needle free” delivery systems. Preferably, the compositions are administered by intradermal injection. Administration can be at the direction of a physician or physician assistant.

The injections can be split into multiple injections, with such split inoculations administered preferably substantially concurrently. When administered as a split inoculation, the dose of the immunogen is preferably, but not necessarily, proportioned equally in each separate injection. If an adjuvant is present in the vaccine composition, the dose of the adjuvant is preferably, but not necessarily, proportioned equally in each separate injection. The separate injections for the split inoculation are administered substantially proximal to each other on the patient's body in some aspects. In some aspects, the injections are administered at least about 1 cm apart from each other on the body. In some aspects, the injections are administered at least about 2.5 cm apart from each other on the body. In some aspects, the injections are administered at least about 5 cm apart from each other on the body. In some aspects, the injections are administered at least about 10 cm apart from each other on the body. In some aspects, the injections are administered more than 10 cm apart from each other on the body, for example, at least about 12.5. 15, 17.5, 20 cm, or more cm apart from each other on the body. Primary immunization injections and booster injections can be administered as a split inoculation as described and exemplified herein.

Various alternative pharmaceutical delivery systems can be employed. Non-limiting examples of such systems include liposomes and emulsions. Certain organic solvents such as dimethylsulfoxide also can be employed. Additionally, the vaccine compositions can be delivered using a sustained-release system, such as semipermeable matrices of solid polymers containing the therapeutic agent. The various sustained-release materials available are well known by those skilled in the art. Sustained-release capsules can, depending on their chemical nature, release the vaccine compositions over a range of several days to several weeks to several months.

In order to prevent cancer recurrence in a patient who is in cancer remission, a therapeutically effective amount of the vaccine composition is administered to the subject. A therapeutically effective amount will provide a clinically significant increase in the number of E75-specific cytotoxic T-lymphocytes (CD8⁺) in the patient, as well as a clinically significant increase in the cytotoxic T-lymphocyte response to the antigen, as measured by any means suitable in the art. In the patient on the whole, a therapeutically effective amount of the vaccine composition will destroy residual microscopic disease and significantly reduce or eliminate the risk of recurrence of cancer in the patient.

The effective amount of the vaccine composition can be dependent on any number of variables, including without limitation, the species, breed, size, height, weight, age, overall health of the patient, the type of formulation, the mode or manner or administration, or the presence or absence of risk factors that significantly increase the likelihood that the cancer will recur in the patient. Such risk factors include, but are not limited to the type of surgery, status of lymph nodes and the number positive, the size of the tumor, the histologic grade of the tumor, the presence/absence of hormone receptors (estrogen and progesterone receptors), HER2/neu expression, lymphovascular invasion, and genetic predisposition (BRCA 1 and 2). In some preferred aspects, the effective amount is dependent on whether the patient is lymph node positive of lymph node negative, and if the patient is lymph node positive, the number and extent of the positive nodes. In all cases, the appropriate effective amount can be routinely determined by those of skill in the art using routine optimization techniques and the skilled and informed judgment of the practitioner and other factors evident to those skilled in the art. Preferably, a therapeutically effective dose of the vaccine compositions described herein will provide the therapeutic preventive benefit without causing substantial toxicity to the subject.

Toxicity and therapeutic efficacy of the vaccine compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀ /ED₅₀. Vaccine compositions that exhibit large therapeutic indices are preferred. Data obtained from cell culture assays and animal studies can be used in formulating a range of dosage for use in patients. The dosage of such vaccine compositions lies preferably within a range of circulating concentrations that include the ED₅₀ with little or no toxicity. The dosage can vary within this range depending upon the dosage form employed and the route of administration utilized.

Toxicity information can be used to more accurately determine useful doses in a specified subject such as a human. The treating physician can terminate, interrupt, or adjust administration due to toxicity, or to organ dysfunctions, and can adjust treatment as necessary if the clinical response is not adequate, to improve the response. The magnitude of an administrated dose in the prevention of recurrent cancer will vary with the severity of the patient's condition, relative risk for recurrence, or the route of administration, among other factors. The severity of the patient's condition can, for example, be evaluated, in part, by standard prognostic evaluation methods.

The vaccine compositions can be administered to a patient on any schedule appropriate to induce and/or sustain protective immunity against cancer relapse, and to induce and/or sustain a cytotoxic T lymphocyte response. For example, patients can be administered a vaccine composition as a primary immunization as described and exemplified herein, followed by administration of a booster to bolster and/or maintain the protective immunity. Patients can be administered the vaccine compositions 1, 2 or more times per month.

The vaccine administration schedule, including primary immunization and booster administration, can continue as long as needed for the patient, for example, over the course of several years, to over the lifetime of the patient. In some aspects, the vaccine schedule includes more frequent administration at the beginning of the vaccine regimen, and includes less frequent administration (e.g., boosters) over time to maintain the protective immunity.

The vaccine composition can be administered at lower doses at the beginning of the vaccine regimen, with higher doses administered over time. The vaccines can also be administered at higher doses at the beginning of the vaccine regimen, with lower doses administered over time.

The frequency of primary vaccine and booster administration and dose of antigen administered can be tailored and/or adjusted to meet the particular needs of individual patients, as determined by the administering physician according to any means suitable in the art.

The vaccine composition according to an aspect of the present invention is a composition commonly comprising the above-mentioned lipid nanoparticles, and in order to avoid the complexity of the specification, the description thereof is omitted within the overlapping range.

In one embodiment of the present invention, the vaccine composition is for cancer prevention. When the vaccine composition is for cancer prevention, the antigen of the nanolipid particles as an active ingredient is a tumor-associated antigen.

As used herein, the term “prevent” refers to any success or indicia of success in the forestalling of breast cancer recurrence/relapse in patients in clinical remission, as measured by any objective or subjective parameter, including the results of a radiological or physical examination.

In yet another embodiment of the present invention, there is provided a cancer vaccine kit which comprises a lipid nanoparticle including a tumor-associated antigen, a phospholipid, a cationic lipid, and an anionic drug as a first vaccine composition; and comprises the lipid nanoparticle and an immune checkpoint inhibitor as a second vaccine composition,

As used herein, the term “immune checkpoint” refers to a modulator of the immune system. Immune checkpoint molecules include stimulatory immune checkpoint molecules and inhibitory immune checkpoint molecules. Inhibitory checkpoint molecules are targets for cancer immunotherapeutic agents. The inhibitory immune checkpoint molecules include A2AR, B7-H3, B7-H4, BTLA, CTLA-4, IDO, KIR, LAG3, NOX2, PD-1, TMI-3, VISTA, SIGLEC7, and the like, but are limited thereto. The checkpoint inhibitors approved to date target CTLA4, PD-1, and PD-L1.

In one embodiment of the present invention, the checkpoint inhibitor is an anti-PD-1 antibody or an anti-PD-L1 antibody.

### Advantageous Effects

The present invention provides a lipid nanoparticle comprising an antigen, a phospholipid, a cationic lipid, and an adjuvant, a vaccine composition comprising the same, and a cancer vaccine kit. Lipid nanoparticles according to the present invention can easily deliver antigens and anionic drugs into cells. Particularly, a cancer vaccine kit according to the present invention including lipid nanoparticles according to the present invention as a first vaccine composition and lipid nanoparticles and an immune checkpoint inhibitor as a second vaccine composition can be used to effectively suppress tumor regrowth and recurrence triggered by the occurrence of immunosuppression against a cancer nanovaccine.

### BRIEF DESCRIPTION OF THE DRAWINGS

FIG. 1a shows the synthesis method of monoarginine-cholesterol (MA-Chol) used in the present invention.

FIG. 1b shows a 1H-NMR spectrum of MA-Chol in DMSO-d⁶-.

FIG. 1c shows the MALDI-TOF mass spectrum of MA-Chol.

FIG. 2a shows the conjugation method of DSPE-PEG₂₀₀₀-OVA_{PEP} used in the present invention.

FIG. 2b shows the purification results using HPLC of DSPE-PEG₂₀₀₀-OVA_{PEP}.

FIG. 2c shows a MALDI-TOF mass spectrum of DSPE-PEG₂₀₀₀-OVA_{PEP}.

FIG. 3a shows the expected structure of the OVA_{PEP}-SLNP@CpG nanoparticles of the present invention.

FIG. 3b shows the expected action principle of the OVA_{PEP}-SLNP@CpG nanoparticles of the present invention.

FIG. 4 is a figure which evaluates the loading efficiency of CPG-ODN. Specifically, the CpG ODN loading efficiency was evaluated through a Sepharose CL-4B size exclusion column. When 1.65 nmol of CpG ODN was loaded into 8 μmol of OVA_{PEP}-SLNP, the loading efficiency of CpG ODN was almost 100%.

FIG. 5 shows the electron micrographs (TEM) of OVA_{PEP}-SLNP@CpG nanoparticles of the present invention, and their diameters.

FIG. 6 shows the results of measuring the hydrodynamic diameter and zeta potential of the nanoparticles of the present invention by dynamic light scattering (DLS).

FIG. 7 is a figure which has evaluated the cytotoxicity to dendritic cells (DC2.4) of the OVA_{PEP}-SLNP@a CpG nanoparticles of the present invention by WST-1 analysis.

FIG. 8 is a figure which has evaluated intracellular uptake of OVA_{PEP}-SLNP©CpG nanoparticles of the present invention in dendritic cells and bone marrow-derived DCs using the flow cytometry. Rhodamine dye-labeled OVA_{PEP}-SLNP@CpG was used for flow cytometry.

FIG. 9 is a figure which has observed with a confocal laser scanning microscope to confirm the intracellular absorption of the OVA_{PEP}-SLNP©CpG nanoparticles of the present invention.

FIG. 10 is a figure which shows the frequency of mature dendritic cells by treatment with OVA_{PEP}-SLNP@CpG nanoparticles of the present invention.

FIG. 11 shows the expression level of CD80, which is a costimulatory molecule, by treatment with OVA_{PEP}-SLNP@CpG nanoparticles of the present invention.

FIG. 12 shows the expression level of CD86, which is a costimulatory molecule, by treatment with OVA_{PEP}-SLNP@CpG nanoparticles of the present invention.

FIG. 13 shows the B3Z reaction by the treatment with OVA_{PEP}-SLNP@CpG nanoparticles of the present invention.

FIG. 14 is a result of measuring the secretion level of IL-2 by the treatment with OVA_{PEP}-SLNP@CpG nanoparticles of the present invention through ELISA.

FIG. 15 shows the lymphatic drainage of OVA_{PEP}-SLNP@CpG nanoparticles of the present invention. Near-infrared dye-loaded OVA_{PEP}-SLNP@CpG nanoparticles were measured using IVIS.

FIG. 16 shows the lymphatic drainage of OVA_{PEP}-SLNP@CpG nanoparticles of the present invention. The intensity of fluorescence over time after subcutaneous injection was shown.

FIG. 17 shows the in vivo distribution of OVA_{PEP}-SLNP@CpG nanoparticles of the present invention in lymph nodes.

FIG. 18 shows a flow cytometry gating strategy for confirming the distribution of specific cells in lymph nodes. FSC×SSC gating was used to obtain singlets and lymphocytes based on size and presence or absence of granulation, and CD45 was used as a leukocyte marker. CD3⁻CD19⁻7-AAD⁻ cells were gated to exclude T cells, B cells and dead cells.

FIG. 19 shows the uptake of the nanoparticles of the present invention by antigen-presenting cells in lymph nodes through flow cytometry. Rhodamine-labeled OVA_{PEP}-SLNP@CpG was injected into the soles of paws of mice, and popliteal lymph nodes were excised.

FIG. 20 shows a strategy for evaluating the maturity of dendritic cells in lymph nodes by gating CD11c⁺MHCII⁺ cells, which are considered to be mature dendritic cells.

FIG. 21 is the result of evaluating the maturity of dendritic cells in vivo by the treatment with the nanoparticles of the present invention. The maturity markers CD40 and CD86 were measured by flow cytometry.

FIG. 22 shows an immunization schedule for evaluating the in vivo antigen-specific T cell response enhancing effect of the OVA_{PEP}-SLNP@CpG nanovaccine of the present invention.

FIG. 23 shows the level of interferon-gamma secreted from splenocytes after collecting splenocytes from the mice immunized with the OVA_{PEP}-SLNP@CpG nanovaccine and and restimulating the cells by ELISA.

FIG. 24 shows the number of IFN-γ spot forming cells (SFCs) that secrete interferon-gamma from splenocytes after collecting the splenocytes from the mice immunized with the OVA_{PEP}-SLNP@CpG nanovaccine and restimulating the cells by ELISA.

FIG. 25 shows the ratio of CD8+ T cells producing interferon gamma

FIG. 26 shows the ratio of CD8+ T cells producing interferon gamma and granzyme B.

FIG. 27 shows the immunization and experimental schedule of mice used in an in vivo CTL analysis to assess the antigen-specific killing ability of OVA_{PEP}-SLNP@CpG of the present invention.

FIG. 28 is a figure which compares the CTL killing ability quantitatively by measuring CFSE^{high} cells and CFSE^{low} cells to analyze the killing of OVAPEP-specific splenocytes of OVA_{PEP}-SLNP@CpG of the present invention.

FIG. 29 shows the immunization and tumor inoculation schedule for evaluating the tumor antigen-specific tumor preventive effect of OVA_{PEP}-SLNP@CpG of the present invention.

FIGS. 30 and 31 show the average tumor size and tumor size in individual mice after immunizing with OVA_{PEP}-SLNP@CpG of the present invention followed by EL tumor cell inoculation.

FIG. 32 shows the average tumor weight after immunizing with OVA_{PEP}-SLNP@CpG of the present invention followed by EL4 tumor cell inoculation.

FIGS. 33 and 34 show the average tumor size (FIG. 33) and tumor size in individual mice (FIG. 34) after immunizing with OVA_{PEP}-SLNP@CpG of the present invention followed by E.G7-OVA tumor cell inoculation.

FIG. 35 shows the average tumor weight after immunizing with OVA_{PEP}-SLNP@CpG of the present invention followed by E.G7-OVA tumor cell inoculation.

FIG. 36 is a tumor photograph of individual mice after immunizing with OVA_{PEP}-SLNP@CpG followed by E.G7-OVA tumor cell inoculation.

FIG. 37 shows an experimental schedule for evaluating the therapeutic efficacy of OVA_{PEP}-SNP@CpG.

FIGS. 38 and 39 show the average tumor size (FIG. 38) and tumor size in individual mice (FIG. 39) after E.G7-OVA tumor cell inoculation.

FIG. 40 shows the average tumor weight after E.G7-OVA tumor cell inoculation.

FIG. 41 is a tumor photograph of individual mice after E.G7-OVA tumor cell inoculation.

FIG. 42 shows the number of TUNEL-positive cells for removing tumor tissue from mice immunized with OVAPEP-SLNP@CpG of the present invention, and evaluating the anti-tumor efficacy of the nanovaccine of the present invention at a cellular level.

FIG. 43 shows the damaged cells in the tumor tissue of a mouse immunized with OVA_{PEP}-SLNP@CpG of the present invention, which is the tissue stained with H&E.

FIG. 44 shows apoptotic cells in tumor tissues of mice immunized with OVA_{PEP}-SLNP@CpG of the present invention, wherein brown cells represent TUNEL-positive cells.

FIG. 45 shows the number of TUNEL-positive cells counted in three random fields for each group in tumor tissues of mice immunized with OVA_{PEP}-SLNP@CpG of the present invention.

FIG. 46 shows the expression of PD-L1 in tumor tissue through immunohistochemical (IHC) analysis. PD-L1⁺ cells were stained green and the cell nuclei identified by Hoechst staining were show in blue.

FIG. 47 is a figure which has evaluated CD8+ T cell infiltration into tumor tissue through IHC analysis. CD8⁺ T cells were stained red and the cell nuclei identified by Hoechst staining were shown in blue.

FIG. 48 shows the expression level of PD-L1 in E.G7-OVA tumor cells according to the presence or absence of interferon-gamma treatment.

FIG. 49 shows an experimental schedule for evaluating the efficacy of inhibiting tumor recurrence by sequential combination treatment of the OVA_{PEP}-SLNP@CpG nano vaccine and ICP antibody of the present invention.

FIG. 50 shows the gating strategy of mouse PBMC for tetramer analysis, wherein FSC×SSC gating yields singlets and lymphocytes according to size and degree of granulation. CD45 was used as a leukocyte marker and CD3 and CD8 were used as T cell markers. 7-AAD-cells were gated to exclude dead cells, and CD3⁺ CD8⁺ T cells were gated for tetrameric staining analysis.

FIG. 51 shows the results of typical flow cytometry of peripheral blood CD8+ T cells positive for OVAPEP tetramer 20 days after tumor inoculation.

FIGS. 52 and 53 show the percentage of OVAPEP-specific CD8⁺ T cells (FIG. 52) and PD-1⁺ CD8⁺ T cells (FIG. 53) in the peripheral blood of mice immunized with the nanovaccine of the present invention day 20 post after tumor inoculation as determined by flow cytometry (n=6).

FIG. 54 shows the size of a tumor after E.G7-OVA cell inoculation in mice. After the first vaccination cycle, 40 good responders were divided into 4 groups. Poor responders were sacrificed when the tumor volume reached ˜2000 mm³.

FIG. 55 shows the tumor weight at the time of mouse sacrifice for each group. Data are expressed as mean±S.E.M.

FIG. 56 is a figure which visually shows the order and time of using the OVA_{PEP}-SLNP@CpG nanovaccine of the present invention in combination with an immune checkpoint therapeutic agent.

### DETAILED DESCRIPTION

Hereinafter, the present invention will be described more specifically with reference to examples. It will be apparent to those skilled in the art that these examples are for illustrative purposes only, and the scope of the present invention is not limited by these examples in accordance with the gist of the present invention.

### EXAMPLE

Throughout this specification, “%” used to indicate the concentration of a specific substance is (weight/weight) % for solid/solid, (weight/volume) % for solid/liquid and (volume/volume) % for liquid/liquid, unless otherwise stated.

### Experimental Materials and Methods

### Experimental Materials

1,2-Distearoyl-sn-glycero-3-phosphoethanolamine-N-[carboxy(polyethylene glycol)-1000](DSPE-PEG₁₀₀₀), 1,2-distearoyl-sn-glycero-3-phosphoethanolamine-N-[PDP(polyethylene glycol)-2000](DSPE-PEG₂₀₀₀-PDP), 1,2-dioleoyl-sn-glycero-3-phosphoethanolamine (DOPE) and 1,2-dipalmitoyl-sn-glycero-3-phosphoethanolamine-N-(lissamine rhodamine B sulfonyl) (DPPE-Rhodamine) were purchased from Avanti Polar Lipids (Alabaster, Ala., USA). Boc-Arg(Pbf)-OH and cholesterol were purchased from Sigma Aldrich (St. Louis, Mo., USA). CpG oligodeoxynucleotide (CpG ODN; 5′-TCC ATG ACG TTC CTG ACG TT-3′) and control ODN (5′-TCC ATG AGC TTC CTG AGC TT-3′) were synthesized using a phosphorothioate backbone by Genotech (Daejeon, Korea). OVA₂₅₇₋₂₆₄ SIINFEKL (OVAPEP) and SIINFEKL (C-OVA_{PEP}) peptide with N-terminal cysteine were synthesized by Cosmo Genetec (Seoul, Korea). All other reagents were purchased from Sigma Aldrich unless otherwise indicated.

### Animals and Cells

Female C57BL/6 mice were obtained from Orient Bio (Korea) and housed under pathogen free conditions. The animal care and experimental procedures have been approved by the Animal Care and Use Committee of the Korea Advanced Institute of Science and Technology (KAIST). The DC2.4 murine dendritic cell line was provided by Dr. K. L. Rock (University of Massachusetts Medical School, Worcester, Mass., USA). The B3Z murine CD8⁺ T hybridoma cell line was provided by Professor Yongtaek Lim (Sungkyunkwan University). DC2.4 and 83Z cells were maintained using RPMI-1640 medium (WELGENE, Geongsan-si, Korea) supplemented with 10% heat-inactivated fetal bovine serum (FBS; Welgene), 1% penicillin/streptomycin, 2 mM L-glutamine, 10 mM HEPES, 1 mM sodium pyruvate, 1× non-essential amino acid and 50 μM 2-mercaptoethanol. EL4 murine lymphoma cell line, and E.G7-OVA murine EL4 lymphoma cell line transfected with Ovalbumin were purchased from ATCC (American Type Culture Collection; Manassas, Va., USA). EL4 cells were grown in RPMI-1640 medium supplemented with 10% FBS, 1% penicillin/streptomycin, 2 mM L-glutamine, 4.5 g/L glucose, 10 mM HEPES, 1 mM sodium pyruvate and 50 μM 2-mercaptoethanol. E.G7-OVA cells were grown in RPMI-1640 medium supplemented with 10% FBS, 1% penicillin/streptomycin, 2 mM L-glutamine, 4.5 g/L glucose, 10 mM HEPES, 1 mM sodium pyruvate, 50 μM 2-mercaptoethanol and 0.5 mg/mL G418 (Gibco, Grand Island, N.Y., USA). All cells were maintained at 37° C. in a humidified atmosphere containing 5% CO₂.

### Flow Cytometry

All antibodies were purchased from BioLegend (San Diego, Calif., USA), eBiosciences (San Diego, Calif., USA) and Tonbo Biosciences (San Diego, Calif., USA). Antibodies used included anti-CD16/CD32 (clone 2.4G2), anti-CD45 (clone 30-F11), anti-CD3 (clone 145-2C11), anti-CD8 (clone 53-6.7), anti-CD19 (clone 1D3), anti-CD169 (clone 3D6.112), anti-CD11b (clone M1/70), anti-CD11c (clone N418), anti-MHC II (clone M5/114.15.2), anti-CD40 (clone 3/23), anti-CD80 (clone 16-10A1), anti-CD86 (clone GL-1), anti-IFN-y (clone XMG1.2), anti-Granzyme B (clone 16G6), anti-PD-1 (clone 29F.1A12), and anti-PD-L1 (clone 10F.9G2). Cells were blocked with anti-CD16/CD32 antibody at 4° C. for 10 minutes, and immunostained with different antibodies at 4° C. for 20 to 30 minutes. Dead cells were excluded by staining with 7-AAD viability staining solution (BioLegend) or Ghost Dye™ Violet 450 (Tonbo Biosciences). Flow cytometry was performed using a LSRFortessa flow cytometer (BD Biosciences, San Jose, Calif., USA), and data were analyzed using FlowJo software (TreeStar),

### Synthesis of OVA_{PEP}-Phospholipid Conjugate

The C-OVAPEP peptide was conjugated to DSPE-PEG₂₀₀₀-PDP by a disulfide exchange reaction. Briefly, 2 mg of C-OVAPEP and 7.8 mg of DSPE-PEG₂₀₀₀PDP were dissolved in 200 μl DMSO and the solution was gently vortexed overnight at room temperature.

200 μl of acetonitrile was added thereto, and the mixture was quenched, and purified by high performance liquid chromatography (HPLC, Agilent) using a C4 column (Nomura Chemical), The product-containing fraction was lyophilized to give a conjugate (DSPE-PEG2000-OVAPEP) as a white solid. The conjugate was further analyzed by matrix-assisted laser desorption/ionization-time-of-flight (MALDI-TOF) m as spectroscopy (Bruker).

### Preparation and Characterization of OVA_{PEP}-SLNP@CpG Nanovaccine

Monoarginine-cholesterol (MA-Chol) was synthesized as described above (Lee, J. et al. Theranostics 6, 192-203, 2016). A nanovaccine (OVA_{PEP}-SLNP@CpG) based on small lipid nanoparticles (SLNP) was prepared by film formation and rehydration. Briefly, MA-Chol (3.89 μmol), DOPE (3.89 μmol), DSPE-PEG1000 (0.2 μmol) and DSPE-PEG2000-OVAPEP (0.02 μmol) were added to a glass vial and dried overnight under vacuum to completely remove the residual solvent. The resulting lipid film was rehydrated with 1 ml of HEPES-buffered glucose (HBG) containing 1.65 nmol of CpG ODN. The solution was sonicated for 10 minutes, then stirred with a magnetic bar at room temperature for at least 4 hours, and extruded at least 11 times using a small extruder (Avanti Polar Lipids). The morphology and size of OVA_{PEP}-SLNP@CpG was evaluated by transmission electron microscopy (TEM) with 1% uranylacetate solution for negative staining. The average size of the nanoparticles was measured using ImageJ software (National Institutes of Health), and the hydrodynamic size and zeta potential thereof were measured at ambient temperature by dynamic light scattering (DLS) using a Zetasizer Nano range system (Malvern, Worcestershire, UK). The efficiency of CpG ODN loading was evaluated using a Sepharose CL-4B size exclusion column (Sigma Aldrich).

OVA_{PEP}-SLNP@CpG was loaded onto the column washed with HEPES-buffered saline (HBS); 15 eluted fractions were collected, each CpG ODN was measured using Quant-iT OliGreen ssDNA reagent (Thermo Fisher). The loading efficiency of CpG ODN was determined by mixing 100 μl of each fraction with 20 μl of 5% Triton-X 100 and 100 μl of OliGreen, and measuring the fluorescence intensity at an excitation wavelength of 480 nm and an emission wavelength of 520 nm. Fractions 2-4 contained the nanovaccine, but fractions 6-10 contained free CpG ODN due to their size differences. The loading efficiency of 1.65 nmol of CpG ODN in 8 μmol of SLNP was almost 100%.

### Cell Viability Assays

The cytotoxicity of the nanovaccine was assessed by analysis of water-soluble tetrazolium salt (WST-1) using the EZ-Cytox Cell Viability Assay kit (DoGenBio, Seoul, Korea) according to the manufacturer's instructions. Briefly, DC2.4 cells were seeded into 96-well plates at a density of 1×10⁴ cells per well in 100 μl medium, and incubated overnight at 37° C. The cells were treated with OVA_{PEP}-SLNP@CpG and incubated at 37° C. for 24 hours. 10% volume of WST-1 reagent was added to each well and the plates were incubated at 37° C. for 4 hours, Absorbance was measured at 450 nm with a microplate reader (VERASmax™, Molecular Devices).

In Vitro Uptake of Dendritic Cells (DCs)

Intracellular uptake of the OVA_{PEP}-SLNP@CpG nanovaccine was assessed by flow cytometry and confocal laser scanning microscopy. Briefly, DC2.4 cells were seeded into 6-well plates at a density of 5×10⁵ cells per well in 2 ml medium, and allowed to adhere overnight. To detect the intracellular uptake of the nanovaccine, 0.5 wt % of DPPE-rhodamine dye was added to the lipid nanoparticle formulation. Cells were incubated with 200 μM of rhodamine-labeled nanovaccine for 4 hours, and washed with PBS. Cell uptake was assessed by flow cytometry. To confirm cellular uptake by confocal microscopy, DC2.4 cells were seeded at a density of 4×10⁴ cells per well in 0.5 ml medium on coverslips of 24-well plates, grown and adhered overnight. Cells were incubated with 200 μM rhodamine-labeled nanovaccine for 4 hours, washed with PBS, and fixed with 10% formalin solution, and their nuclei were stained with DRAQ5 (Thermo Asher). All samples were imaged by a confocal laser scanning microscope (LSM 780; Carl Zeiss).

### Generation, Uptake, Maturation and T Cell Cross-Priming of BMDCs

BMDC was produced as described in Kang, S. et al. (J Control Release 256, 56-67, 2017). To assess the intracellular uptake of nanovaccines, BMDCs were seeded into 12-well plates ata density of 3×10⁵ cells per well in 0.5 ml medium and allowed to adhere overnight. After incubation with 200 μM Rhodamine-labeled nanovaccine for 4 hours, cells were washed, harvested and stained with anti-CD11c-PE/Cy7 and anti-MHCII-APC antibodies,

Cell uptake was assessed by flow cytometry. To assess the ability of nanovaccines to enhance DC maturation, immature BMDCs were cultured in 12-well plates at a density of 5×10⁵ cells per well in 0.5 ml medium and allowed to adhere overnight. BMDCs were cultured in HBG buffer, soluble CpG, soluble OVA_{PEP}, soluble OVA_{PEP}+CpG, OVA_{PEP}-SLNP@ODN or OVA_{PEP}-SLNP@CpG (CpG: 0.1 μM; OVA_{PEP}: 1.2 μM; SLNP: 0.48 mM) for 24 hours. Then, BMDC was washed, harvested, stained with anti-CD11c-PE/Cy7, anti-MHC±anti-CD80-FITC and anti-CD86-PE antibodies, and analyzed by flow cytometry.

To evaluate the cross-priming of T cells, BMDCs were seeded into 12-well plates at a density of 1×10⁶ cells per well in 1 ml medium and allowed to adhere overnight. BMDCs were incubated with HBG buffer, soluble CpG, soluble OVA_{PEP}, soluble OVA_{PEP}+CpG, OVA_{PEP}-SLNP@ODN or OVA_{PEP}-SLNP CpG (CpG: 0.1 μM; OVA_{PEP}: 1.2 μM; SLNP: 0.48 mM) for 18 hours, harvested, and washed with citrate-phosphate buffer (pH 3.2) on ice for 3 minutes. Peptide/MHC class I complexes were removed from the surface,

These BMDCs were then co-cultured with B3Z CD8⁺ T hybridoma cells for 24 hours. Briefly, BMDCs were seeded into 96-well U-bottom plates at a density of 2×10⁴cells per well in 0.1 ml buffer, and then B3Z cells were added to each well at a density of 4×10⁴ cells per well in 0.1 ml medium and cultured for 24 hours. The suspension was centrifuged to isolate the cell pellet and supernatant.

β-galactosidase activity was assayed on cell pellets. Briefly, the harvested cell pellet was washed and resuspended in CPRG assay buffer (PBS containing 0.1% Triton X-100, 100 μM 2-mercaptoethanol, 10 mM MgCl₂ and chlorophenol red-3-D-galactopyranoside (CPRG)). Each resuspended pellet was transferred to a well of a 96-well plate, and plates were incubated in the dark at 37° C. for 3 hours. The absorbance of each well at 570 nm was measured using a microplate reader, IL-2 concentration in the collected supernatant was assessed using an IL-2 ELISA kit (R&D Systems, Minneapolis, USA) according to the manufacturer's instructions.

### In Vivo Lymphatic Drainage, Uptake and DCs Maturation

To assess the lymphatic drainage of OVA_{PEP}-SLNP@CpG nanovaccine, 037 wt % of pegylated cypate dye was added to the nanoparticle formulation. The pegylated cypate-loaded nanovaccine was injected subcutaneously into the paw soles of C57BL/6 mice. After 2, 4, 8 and 12 hours, the fluorescence signal was assessed using an in vivo imaging system (IVIS). Lymphatic drainage was also assessed by confocal microscopy. Rhodamine dye-labeled nanovaccine was subcutaneously injected into the paw soles of mice, and popliteal LNs were removed after 8 hours. The removed LN was implanted in OCT compound (Leica, Germany) and frozen, and divided into 15 μm slices using a frozen microtome (CM1850; Leica), which was mounted on a glass slide. LN sections were fixed with 10% formalin solution and blocked with PBS containing 2% bovine serum albumin (BSA) for 1 hour at room temperature. Slides were mounted with a VectaMount™ AQ mounting medium (Vector Laboratories, Burlingame, Calif., USA) and imaged by confocal laser scanning microscopy. To confirm uptake by APC in LN, rhodamine-labeled nanovaccine was injected subcutaneously into the paw soles of mice. After 8 hours, the popliteal lymph node LN was removed. The removed LN was washed, excised and digested in collagenase type IV solution (1 mg/ml; Sigma Aldrich) at 37° C. for 30 minutes. The cells were washed again and passed through a 70 μm cell strainer (Falcon) to recover a single cell suspension. LN cells were cultured with anti-CD45-Pacific Blue, anti-CD3-PerCP/Cy5.5, anti-CD19-PerCP/Cy5.5, anti-CD169-F ITC, anti-CD11b-APC, anti-CD11c-PE/Cy7 antibody and with 7-AAD at 4° C. for 20 minutes. These cells were washed and analyzed by flow cytometry.

### Immunization

Six-week-old female C57BU6 mice were immunized using a homologous prime-boost regimen. Mice were divided into four groups, which were injected subcutaneously with HBG buffer vehicle, soluble OVA_{PEP}+CpG, OVA_{PEP}-SLNP ODN, or OVA_{PEP}-SLNP CpG (CpG: 0.4 nmol per mouse; OVA_{PEP}: 5 nmol per mouse; SLNP: 2 μmol per mouse) into both paw soles at time points indicated, and immunized.

### Evaluation of Antigen-Specific T Cell Responses

As mentioned above, mice divided into 4 groups were immunized 3 times at 10-day intervals and sacrificed 3 weeks after the last immunization. To evaluate antigen-specific T cell responses, splenocytes were restimulated ex vivo with OVA_{PEP} (SIINFEKL peptide; 10 μg/ml). The amount of secreted IFN-γ was determined by enzyme-linked immunosorbent assay (ELISA) and the number of INF-γ producing cells was assessed by enzyme-linked immunospot (ELISpot) assay. INF-γ and granzyme B produced by CD8⁺ T cells were quantified by intracellular cytokine staining (ICS). To measure INF-γ levels by ELISA, splenocytes were seeded into 96-well U bottom plates at a density of 3×10⁵ cells per well and restimulated with OVA_{PEP} for 72 hours. The culture supernatant was harvested and the IFN-γ concentration was measured using an IFN-γ ELISA kit (R&D Systems). To measure INF-γ producing cells by ELISpot, splenocytes were seeded into 96-well microplates coated with a monoclonal antibody specific for mouse IFN-γ at a density of 3×10⁵ cells per well, and the cells were restimulated with OVA_{PEP} for 30 hours INF-γ producing spots were developed using a mouse IFN-γ ELISpot kit (R&D Systems) according to the manufacturer's protocol. After development, blue-black spots of cytokine localization sites were counted using an automated ELISpot reader (AID GmbH, Strassberg, Germany). For the ICS assay, splenocytes (3×10⁶ cells per round bottom test tube) were restimulated with OVAPEP for 1 hour. To suppress the intracellular transport of cytokines, GolgiStop™ or GolgiPlug™ (BD Biosciences) was added to each tube. The cells were incubated for 5 hours, and stained with Ghost Dye™ Violet 450 at 4° C. for 30 minutes, and the apoptotic cells were identified, and then stained with anti-CD3-PerCP/Cy5.5 and anti-CD8-APC/Cy7 antibodies at 4° C. for 20 minutes. For intracellular cytokine staining, the cells were permeabilized using Cytofix/Cytoperm™ solution (BD Biosciences) and incubated with PE-conjugated anti-IFN-γ and Alexa Fluor 647-conjugated anti-Granzyme B antibodies. Samples were washed and analyzed by flow cytometry.

### In Vivo Cytotoxic T Lymphocyte Assay

As mentioned above, mice were divided into four treatment groups and immunized three times at 7-day intervals. Seven days after the last immunization, mice were injected with a mixture of cells prepared from splenocytes of non-immune C57BL/6 mice. Half of the splenocytes were pulsed with OVA_{PEP} (1 μg/ml) at 37° C. for 1 hour, and the other half was not pulsed. Non-pulsed cells were labeled with 0.5 μM carboxyfluorescein succinimidyl ester (CFSE), and OVAPEP pulsed cells were labeled with 5 μM CFSE for 10 minutes. A 1:1 mixture of pulsed (CFSE^{high}) and non-pulsed (CFSE^{low}) cells was injected intravenously into immunized mice. 18 hours after injection, the splenocytes of recipient mice were harvested and analyzed by flow cytometry. The relative numbers of CFSE^{high} and CFSE^{low} cells were measured. Antigen-specific target targeted apoptosis was calculated using the following Equation:

Specific target apoptosis percentage=100−[100×{(% CFSEhigh immunized mouse/% CFSE^{low} immunized mouse)/(% CFS^{high} non-immunized mouse/%CFSE^{low} N non-immunized mouse)}]

### Antitumor Efficacy Test

To evaluate the therapeutic effect in tumor prevention, mice were immunized three times at 10-day intervals with each vaccine modality described above. 3 Weeks after the last immunization, 2×10⁵EL4 cells were inoculated subcutaneously in one flank of each mouse, and the other side was inoculated subcutaneously with 2×10⁵ E.G7-OVA cells. The tumor growth was monitored every two days using digital calipers, and the tumor volume was calculated as 0.5× length×width², Mice were euthanized when the average tumor volume reached the ethical dead point (˜2000 mm³).

To analyze the effect of treatment in the tumor volume reduction. 2×10⁵ E.G7-OVA cells were subcutaneously inoculated into the right flank of each mouse. When the average tumor volume reached ˜50 mm³, mice were randomly divided into 4 treatment groups and immunized three times at 4-day intervals. To evaluate the efficacy of combination immunotherapy, mice underwent two immunization cycles with and without antibodies to mouse-PD-1 (alpha PD-1; BioXcell; done: RMP1-14). 2×10⁵ E.G7-OVA cancer cells were subcutaneously injected into the right flank and inoculated into mice. The first immunization cycle, consisting of three subcutaneous injections of OVA_{PEP}-SLNP@CpG nanovaccine at 4-day intervals, started after 6 days. On the 20th day, mice were divided into mice with small-tumors (good responders) and mice with large-tumors (poor responders). Poor responders were sacrificed when the tumor volume reached ˜2000 mm³. Good responders started on the 26th day and started the second immunization cycle. The second immunization cycle consisted of two subcutaneous injections at 6-day intervals. Additionally, alpha PD-1 (200 μg per injection) was intraperitoneally administered to mice on days 1, 3 and 5 after each vaccination. When the dead point was reached, the tumor tissue was removed, weighed and photographed.

### In Vitro Induction of PD-L1

E.G7-OVA cells were seeded into 24-well plates at a density of 1×10⁵ cells per well in 0.5 ml medium, Cells were treated with recombinant murine IFN-γ (100 ng/ml; Peprotech, Rocky Hill, N.J., USA) for 48 hours, washed, harvested and stained with PE-conjugated anti-PD-L1 antibody. In vitro induction of PD-L1 was confirmed by flow cytometry.

### Histopathological Analysis of Tumor Tissues

The excised tumor tissue was implanted in OCT solution, immediately frozen, cut into 20 μm slices using a freezing slide, which was mounted on a glass slide. Tissue sections were fixed with 10% formalin solution for 10 minutes, and blocked with PBS containing 2% BSA for 1 hour at room temperature. The tissue sections were incubated with biotin-conjugated anti-mouse CD8a antibody (1:100 dilution; Tonbo Biosciences) overnight at 4° C., so that CD8+ T cell infiltration into tumor tissue was evaluated.

The tissue sections are then washed and incubated with PE-conjugated anti-streptavidin antibody (1:200 dilution; BD Biosciences) at room temperature for 1 hour. The tissue sections were incubated with rat monoclonal anti-PD-L1 antibody (1:100 dilution; Abcam) overnight at 4° C. so that PD-L1 induction in tumor tissue was evaluated. These sections were then washed and incubated with Alexa Fluor 488-conjugated goat anti-rat IgG antibody (1:100 dilution; Abcam) at room temperature for 1 hour.

Nuclei were stained with Hoechst 33342 (1:5000 dilution) and slides were mounted with VectaMount AQ mounting medium, All sections were imaged by confocal laser scanning microscopy. The excised tumor tissue was fixed with 10% formalin solution, and embedded in paraffin and cut into 4 μm slices. These sections were stained with H&E, and apoptotic cells were measured using the Dead End Colorimetric TUNEL system (Promega, Madison, Wis., USA) according to the manufacturer's instructions. AH slides were analyzed using a Nikon upright fluorescence microscope.

### Tetramer Analysis

Mouse peripheral blood obtained by retroorbital bleed was collected in a serum separator tube (BD). Red blood cells (RBC) were removed by incubating with 1 ml of RBC lysis buffer (Biolegend) with gentle shaking at room temperature for 2 minutes. For tetramer staining, blood cells were incubated with iTAg H-2Kb OVA tetramer-PE (MBL, Japan) at 4° C. for 30 minutes. Then, the cells are washed, and incubated with Pacific Blue-conjugated anti-CD45, PE/Cy7 conjugated anti-CD3, Alexa Fluor 488 conjugated anti-CD8, and APC conjugated anti-PD-1 antibodies and 7-AAD at 4° C. for 20 minutes. Cells were washed again and analyzed by flow cytometry.

### Statistical Analysis

Data were expressed as mean±S.E.M. Groups were compared by a one-way analysis of variance (ANOVA) with post hoc Tukey test using GraphPad Prism 5 (GraphPad Software). Statistical significance was defined as P<0.05.

### Example 1: Synthesis and Characterization of Antigen- and Adjuvant-Carrying Nanovaccine (OVA_{PEP}-SLNP@CpG) of the Present Invention

The small lipid nanoparticle (SLNP) used as antigen- and adjuvant-carrying nanovaccine in the present invention were prepared with two phospholipids: i) 1,2-dioleoyl-sn-glycero-3-phosphoethanolamine (DOPE) and ii) 1,2-distearoyl-sn-glycero-3-phosphoethanolamine-[carboxy(polyethylene glycol)1000](DSPE-PEG1000); and a cholesterol derivative: monoarginine-cholesterol (MA-Chol).

The DOPE is a neutral lipid involved in endosome escape of lipid nanoparticles. Therefore, the incorporation of DOPE into SLNPs can enhance antigen migration from endosomes to the cytoplasm to promote antigen expression at the cell membrane. The DSPE-PEG₁₀₀₀ is a PEGylated phospholipid that increases colloidal stability of SLNP under physiological conditions to promote lymphatic drainage of SLNP.

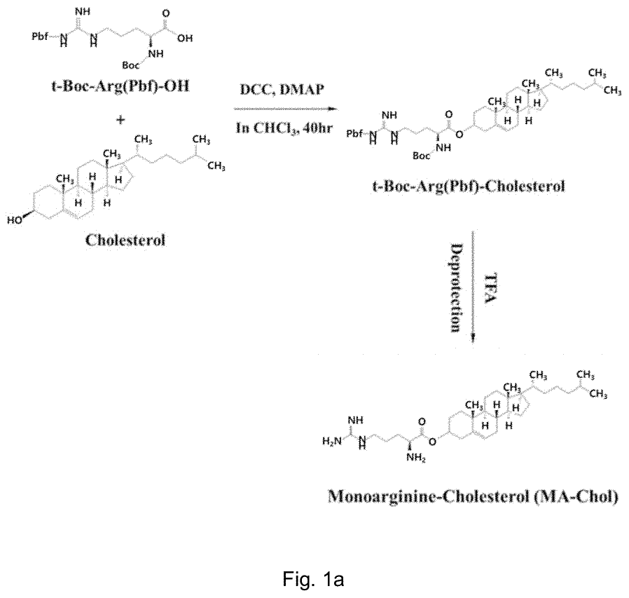

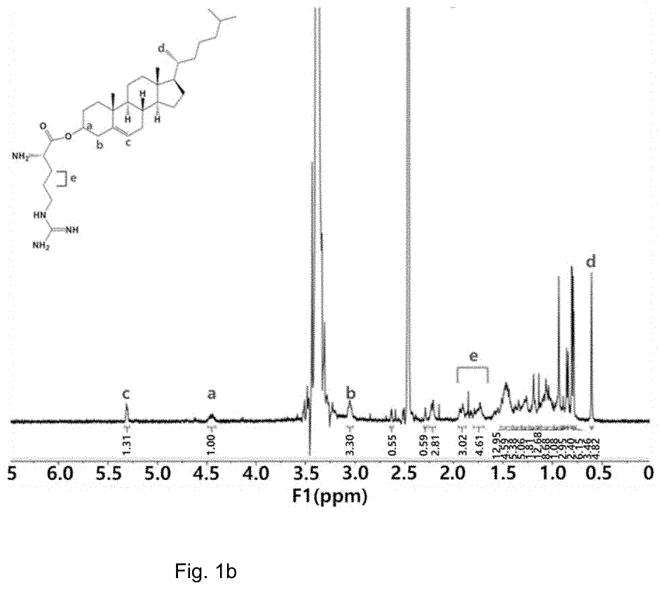

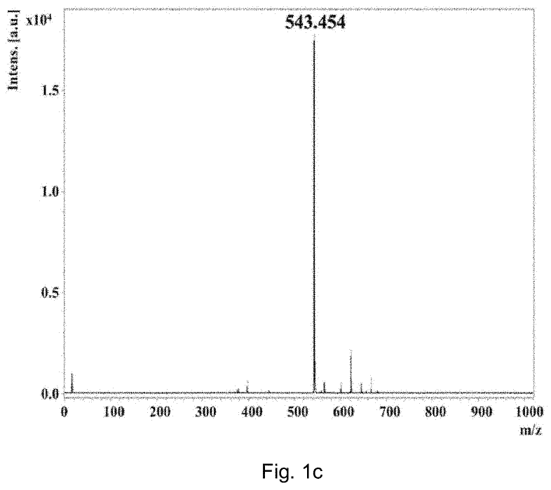

The MA-Chol is a cationic molecule composed of arginine, cholesterol, and major components of SLNP, and enables complex formation between SLNP and oligonucleotides (see FIGS. 1a-1c).

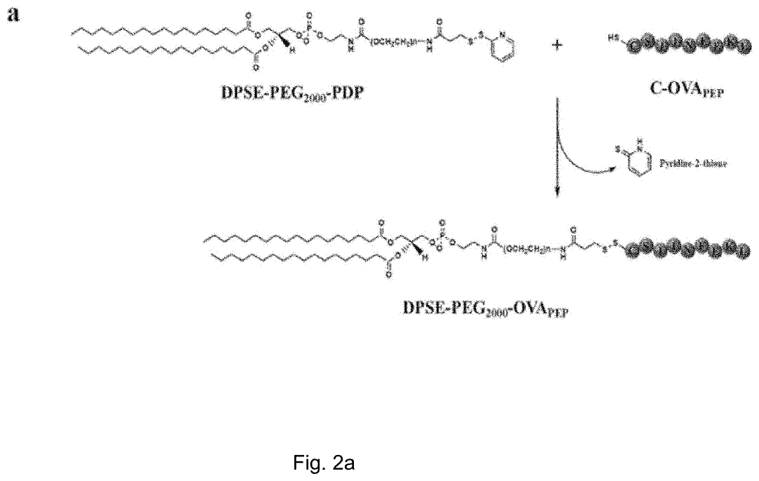

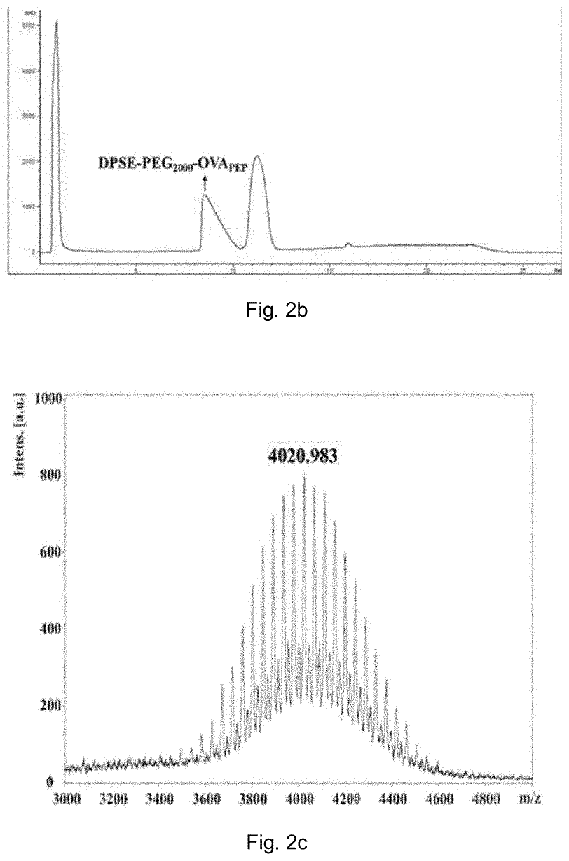

The adjuvant used in the present invention is a toll-like receptor 9 agonistic CpG oligodeoxynucleotide (CpG ODN). The combination of CpG ODN and ICB immunotherapy has been reported to have potent synergistic antitumor efficacy, and several clinical trials of this combination are currently underway. The model tumor antigen was an MHC class I-restricted epitope of ovalbumin (SIINFEKL; named OVAPEP), which has been shown to stimulate CD8⁺ T cell responses. OVA_{PEP} was chemically bound adhered to the end of PEGylated DSPE via a disulfide bond (see FIGS. 2a-2c).

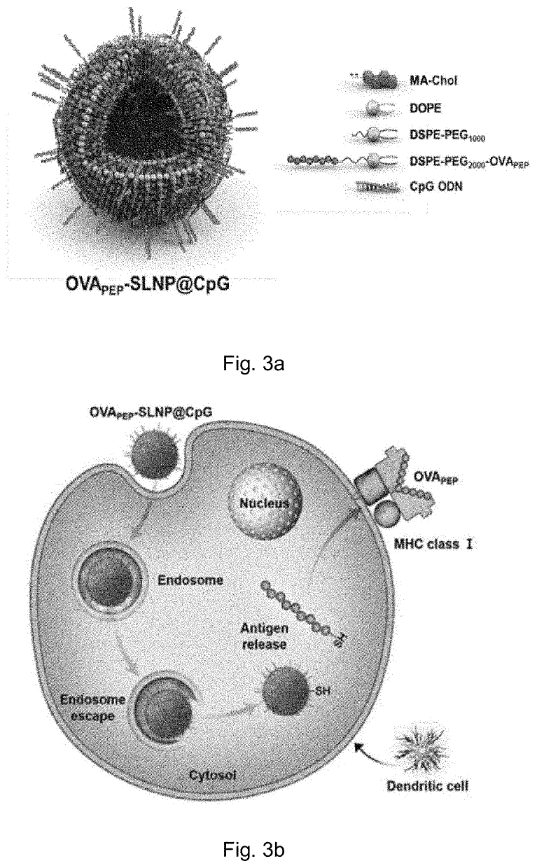

When internalized into cells, this molecule was cleaved by glutathione in the cytoplasm, and the released free OVAPEP was presented to the cell membrane by MHC class I or II (see FIGS. 3a-3b).

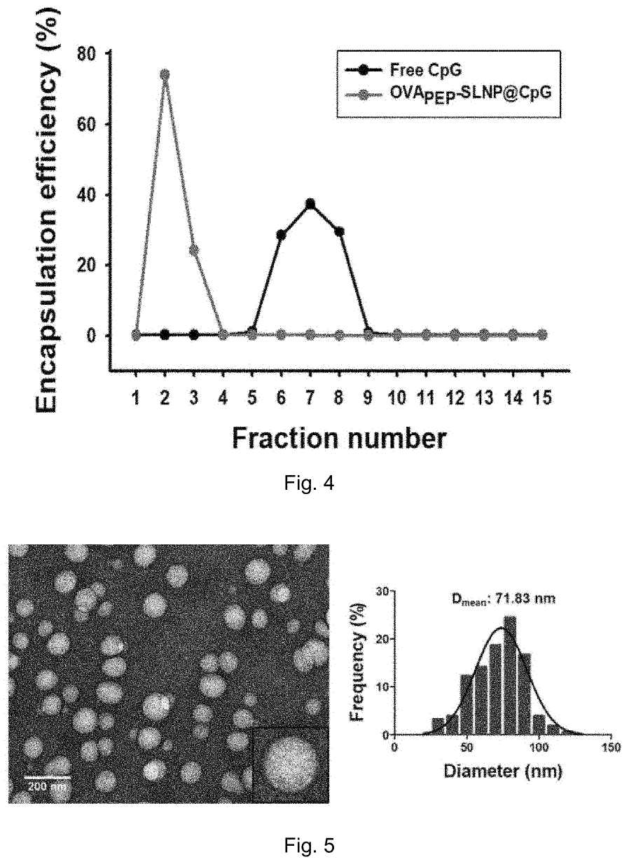

An antigen-labeled, CpG adjuvant-containing SLNP, designated as OVAPEP-SLNP@CpG, was prepared by a one-pot sequential process of film formation and rehydration (see FIG. 1a). Size exclusion chromatography showed almost complete loading of CpG ODN onto OVA_{PEP}-SLNP (see FIG. 4).

The transmission electron microscopy (TEM) showed that OVA_{PEP}-SLNP@CpG, prepared using 0.25 mol % of OVA_{PEP} antigen, had a spherical morphology, and had an average diameter of ˜72 nm by analyzing 155 particles (see FIG. 5).

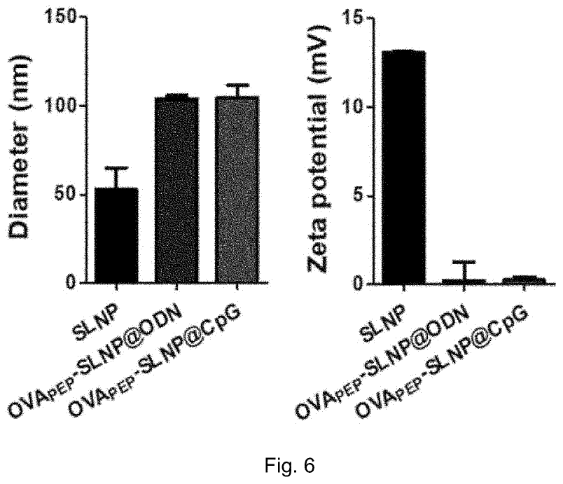

These OVA_{PEP}-SLNP@CpG particles had a hydrodynamic size of ˜104.5 nm and a zeta potential of +0.23 mV, indicating a neutral surface charge. As a control nanovaccine similar in size and zeta potential to OVAPEP-SLNP@CpG, a non-immunostimulant control ODN-conjugated SLNP (OVAPEP-SLNP@ODN) was prepared using the same procedure (see FIG. 6).

### Example 2: In Vitro DC Maturation and T Cell Cross-Priming Enhancing Effect of OVA_{PEP}-SLNP@CpG Nanovaccine of the Present Invention

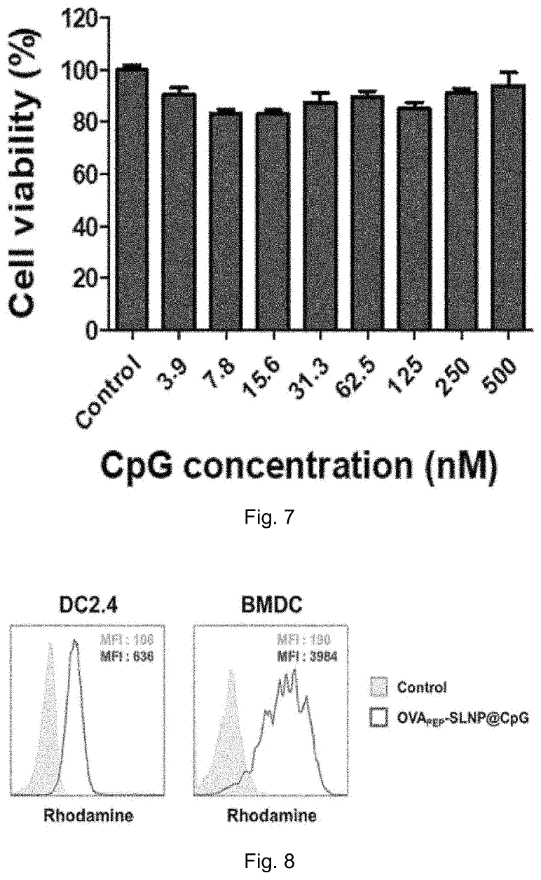

Since the in vivo nanovaccine is expected to be uptaken by dendritic cells (DC) or macrophages, the cytotoxicity of OVA_{PEP}-SLNP@CpG to DCs was evaluated using the WST-1 assay. The nanovaccine of the present invention did not affect the viability of DC2.4 murine DC even at a high CpG concentration of 500 nM (see FIG. 7).

The intracellular uptake of nanovaccines by DCs was evaluated using OVA_{PEP}-SLNP@CpG labeled with rhodamine dye, and the flow cytometry showed a new band of rhodamine-positive cells, which was different from the bands of original DC2.4 cells and bone marrow-derived DCs (BMDCs) (see FIG. 8).

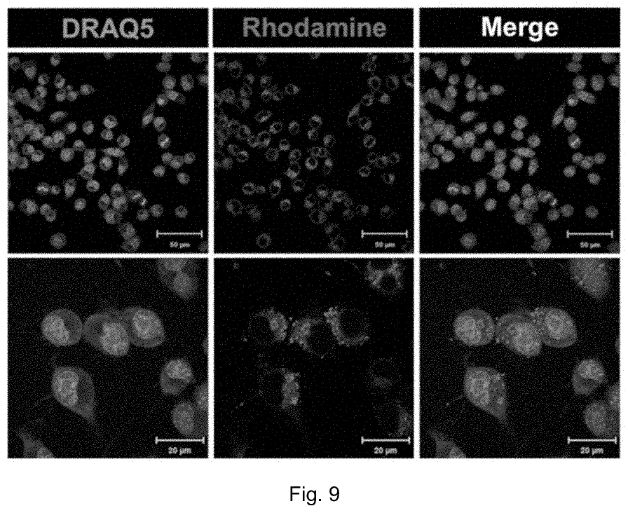

Confocal laser scanning microscopy further confirmed the intracellular localization of the nanovaccine (see FIG. 9).

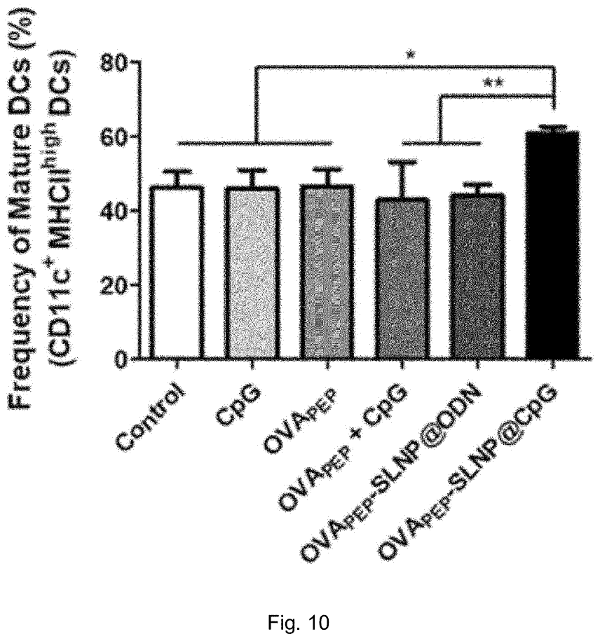

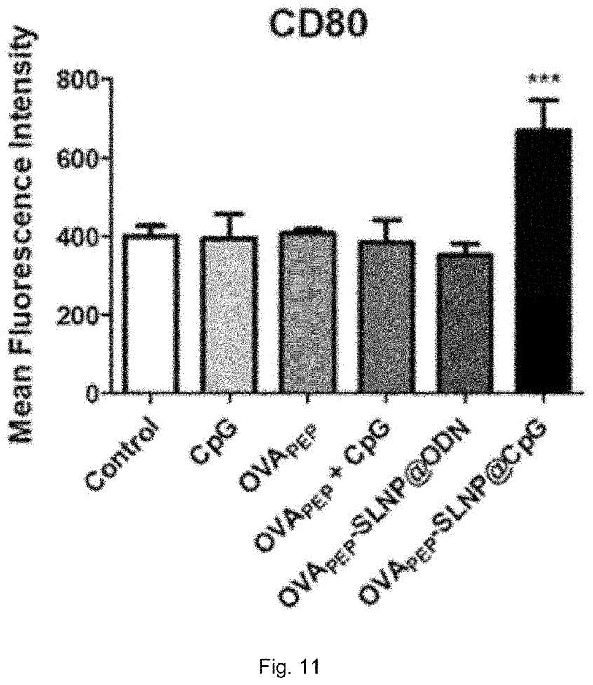

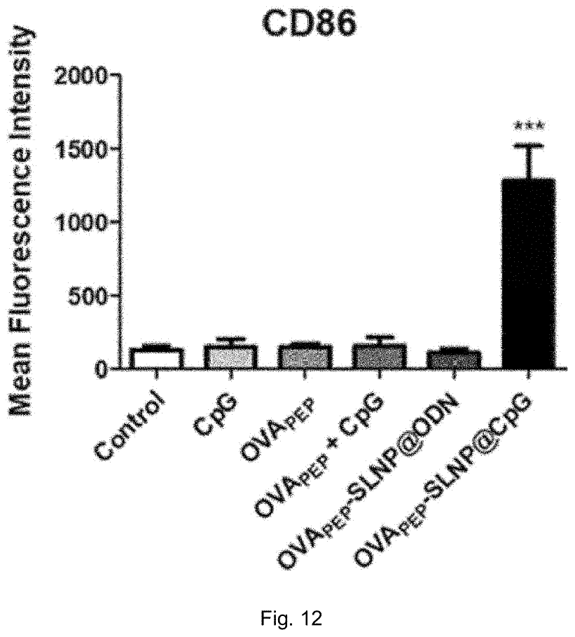

Maturation of DCs and membrane presentation of delivered antigens via MHC class I molecules are essential for inducing an effective CD8+ T cell response. Flow cytometry was performed to evaluate whether OVA_{PEP}-SLNP©CpG could enhance DC maturation. The frequency of mature DCs expressing the marker CD11c⁺ MHCIIhigh and the expression of costimulatory molecules (CD80 and CD86) were significantly higher than in other cell groups (see FIGS. 10˜12).

A mixture of soluble antigen and adjuvant (OVA_{PEP}+CpG CDN) or control OVA_{PEP}-SLNP@ODN did not induce DC maturation, which indicates the importance of the CpG adjuvant. However, soluble CpG alone could not induce DC maturation, which suggests that DC needs an appropriate delivery system. These findings indicate that the OVA_{PEP}-SLNP@CpG nanovaccine can be uptaken by DCs to induce maturation.

When the T cell receptor specifically recognizes the OVA_{PEP} (SIINFEKL)-MHC complex, the cross-priming ability of nanovaccines in BMDC and CD8⁺ T cell hybridoma B3Z cells engineered to secrete β-galactosidase was evaluated. BMDCs were treated with each vaccine modality for 18 hours and washed thoroughly, whereby any antigenic peptides present on the MHC molecule were removed extracellularly so as to avoid intracellular processing and cross-presentation, and co-cultured with B3Z cells for 24 hours.

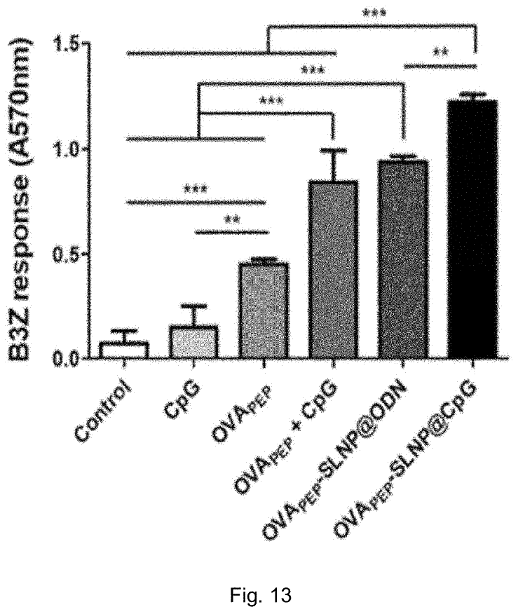

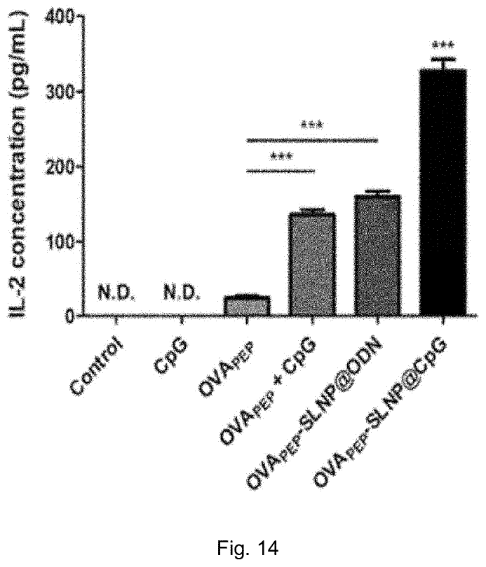

β-galactosidase assay and IL-2 enzyme-linked immunosorbent assays (ELISA) showed that OVA_{PEP}-SLNP@CpG induced significantly higher levels of β-galactosidase and IL-2 secretion than other therapies (see FIG. 2d). Both OVA_{PEP}-SLNPQODN and OVAPEP CpG showed higher activity than soluble OVA_{PEP} or CpG alone (see FIGS. 13 and 14 but was much lower than OVA_{PEP}-SLNP@CpG.

Taken together, the results of these in vitro studies suggest that the OVA_{PEP}-SLNP@CpG nanovaccine is readily uptaken by DCs to induce maturation, and presents the antigen released to the surface via MHC, thereby an effectively cross-priming CD8⁺ T cells to antigen.

### Example 3: Local Injection of OVAPEP-SLNP@CpG Results in Maturation of DCs in Lymph Nodes

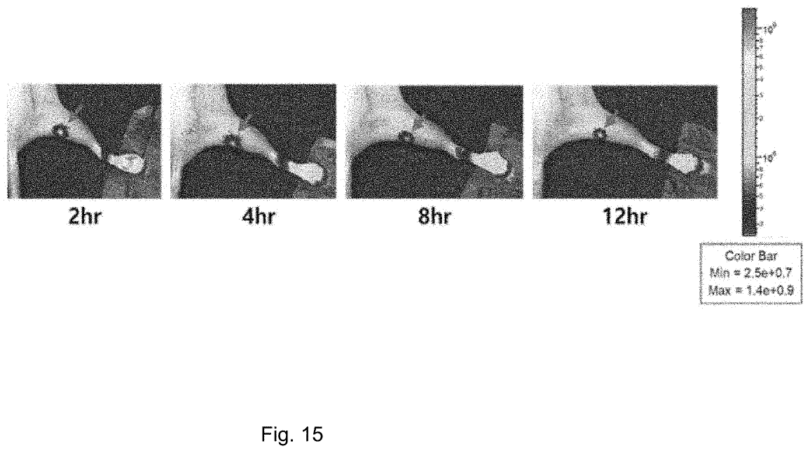

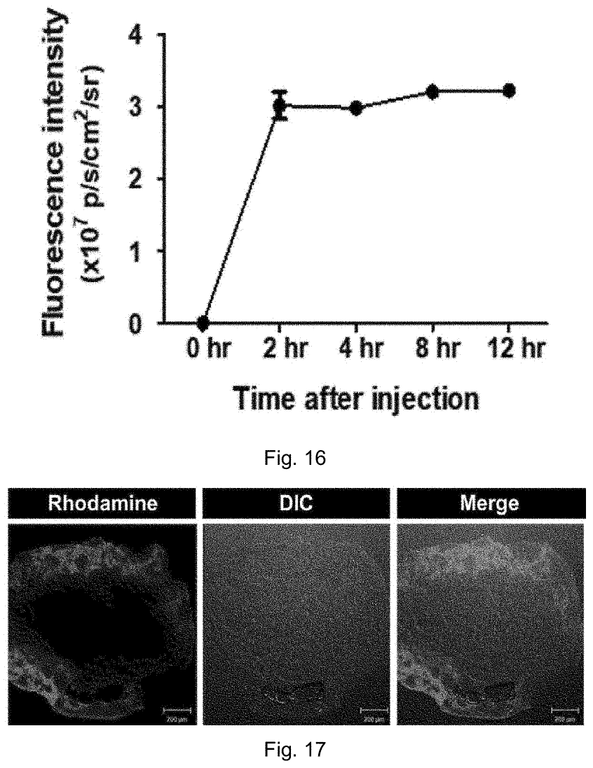

Depending on size and surface function, locally injected nanovaccines have been shown to drain into regional lymph nodes (LNs), wherein the nanovaccine was uptaken by antigen presenting cells (APCs) such as DCs and macrophages. To evaluate the lymphatic drainage of the nanovaccine, the present inventors subcutaneously injected OVAPEP-SLNP@CpG labeled with a near-infrared dye into the paw soles of C57BL 6 mice. The in vivo imaging system (IVIS) showed a clear fluorescence signal intensity around the draining LN, and the fluorescence signal started after 2 hours and lasted for 12 hours (see FIGS. 15-16).

To investigate the distribution of nanovaccine in lymph nodes, OVA_{PEP}-SLNP@CpG labeled with rhodamine dye was injected subcutaneously, and after 8 hours, draining popliteal LN was excised. Confocal microscopy showed that most of the nanovaccines were localized to the subcapsular sinus region of the lymph node (see FIG. 17).

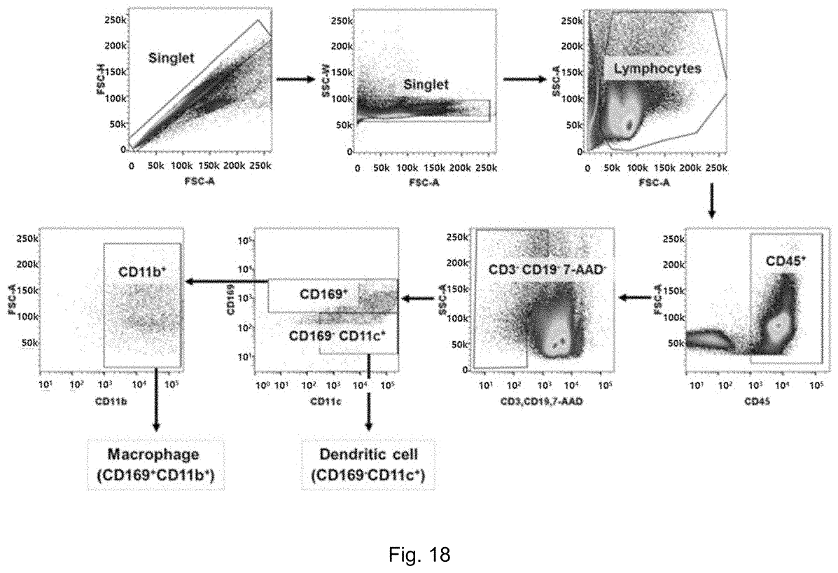

When OVA_{PEP}-SLNP@CpG reaches the nearest lymph node within 2 hours, nanovaccines are more likely to be excreted directly into the LN via lymphatic vessels rather than being uptaken by DCs at the injection site and delivered to the lymph nodes. This process takes ˜24 hours. The ability of APCs to uptake dye-labeled nanovaccine in popliteal lymph nodes was assessed by flow cytometry-based gating (see FIG. 18).

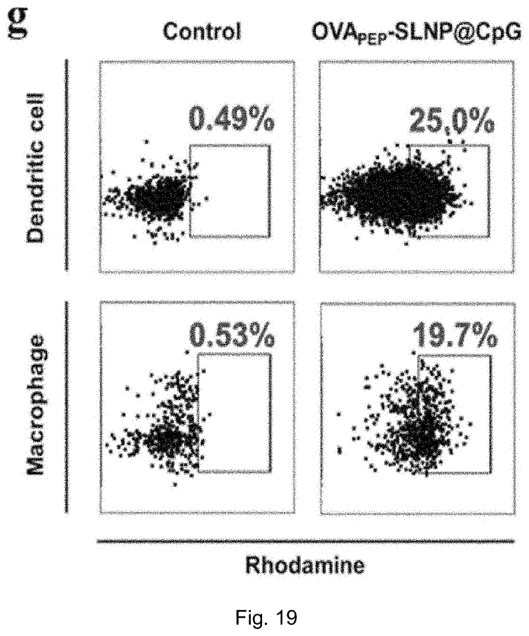

Approximately 19.7% of macrophages (CD169⁺ CD11b⁺) and 25% of DCs (CD169− CD11c⁺) were rhodamine fluorescence-positive, which suggests a high level of nanovaccine uptake by APCs in lymph nodes (see FIG. 19).

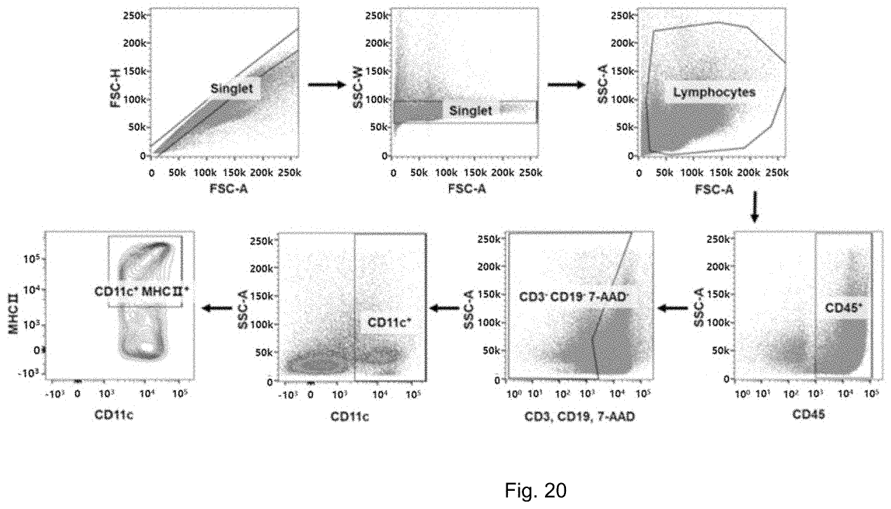

After confirming the ability of OVA_{PEP}-SLNP@CpG of the present invention to activate and promote DC maturation in vitro, the present inventors evaluated DC maturation in popliteal lymph nodes containing nanovaccines by a gating strategy using the maturation markers CD40 and CD86 (see FIG. 20).

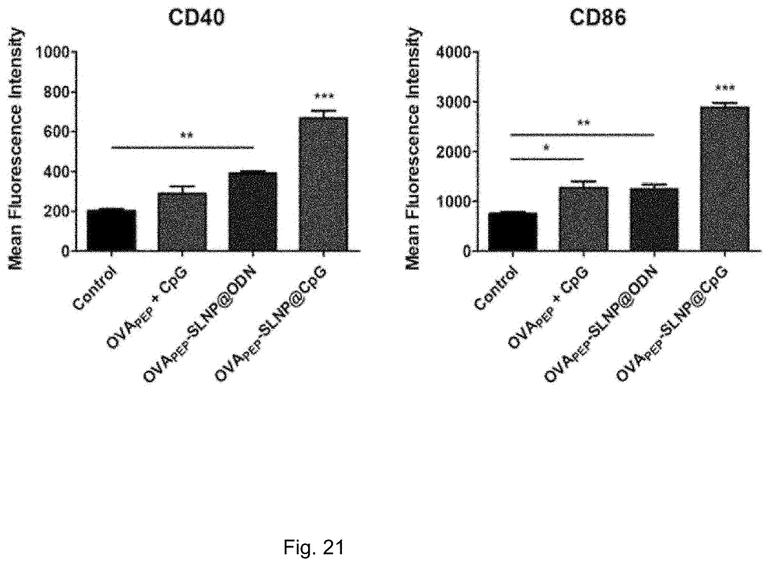

OVA_{PEP}-SLNP@CpG significantly increased the expression of CD40 and CD86, but a mixture of soluble OVAPEP and CpG, or control OVA_{PEP}-SLNP@ODN slightly increased the expression of these markers (see FIG. 21).

Taken together, these in vivo results demonstrated that the OVA_{PEP}-SLNP@CpG nanovaccine of the present invention can be delivered directly to regional lymph nodes with high efficiency, and uptaken by DCs and macrophages residing in lymph nodes, and can effectively induce DC maturation.

### Example 4: Evaluation of Antigen-Specific Cytotoxic T Cell Responses In Vivo

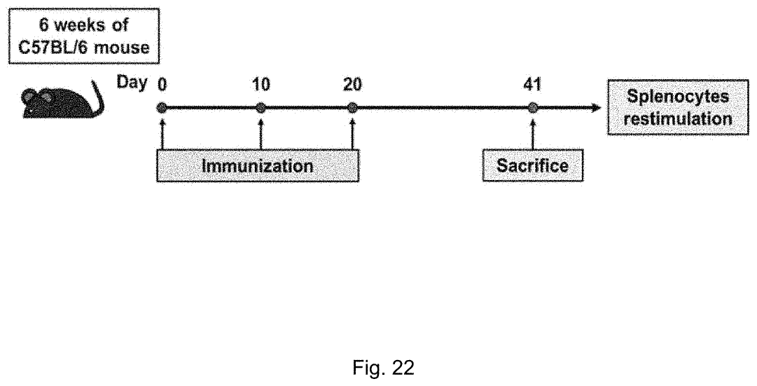

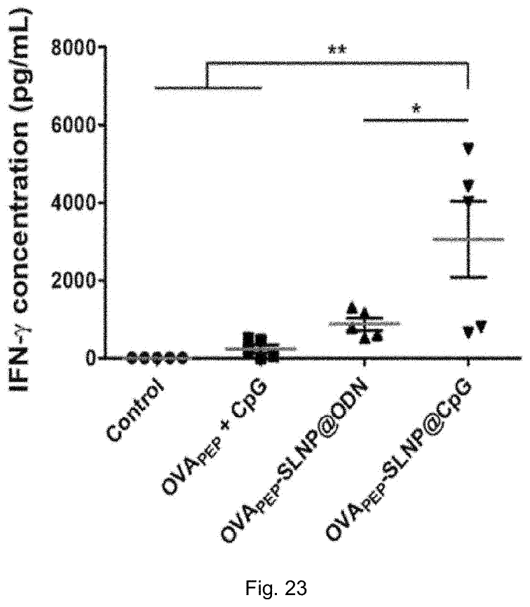

To assess the antigen-specific CD8+ T cell response of i) vehicle, ii) soluble OVA_{PEP}+CpG, iii) OVA_{PEP}-SLNP@ODN or iv) OVA_{PEP}-SLNP@CpG, each substance was immunized to mice three times (0 day, 10 days, 20 days) at 10-day intervals, and sacrificed on the third week (41 days) after the third immunization (see FIG. 22).

After splenocytes were isolated from immunized mice and restimulated with OVA_{PEP} (SIINFEKL peptide), the secretion of interferon-gamma (IFN-γ), which is a representative cytokine secreted by activated CD8⁺ T cells, was measured by ELISA and ELISpot assays.

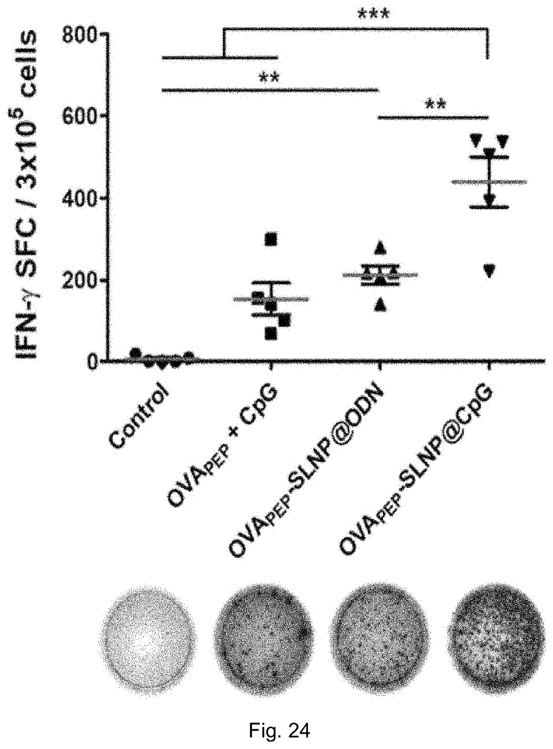

OVA_{PEP}-SLNP@CpG immunization induced greater secretion of INF-γ in ELISA (see FIG. 3b), and in the ELISpot assay, the production of INF-γ spot forming cells (SFC) was much higher than that of other immunogens (see FIG. 24).

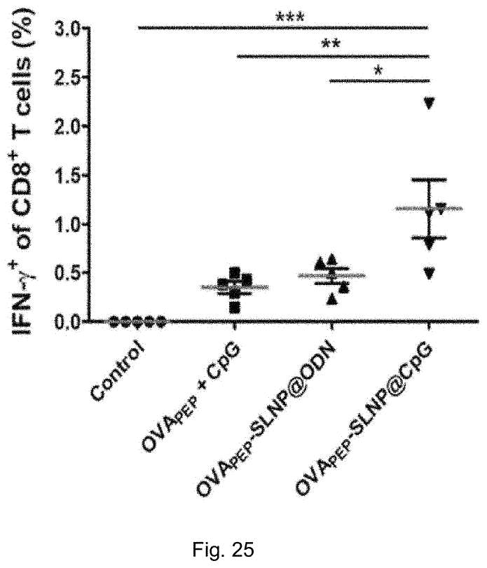

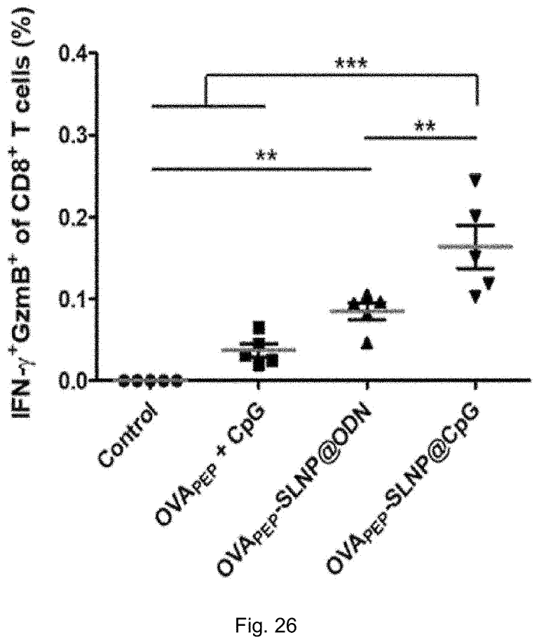

Intracellular cytokine staining (ICS) was performed to test the functionality of activated CD8⁺ T cells, and INF-γ and granzyme B were measured using the gating strategy shown in Supplementary FIG. 6. Soluble OVA_{PEP}+CpG and OVA_{PEP}-SLNP@ODN were ineffective in inducing antigen-specific T cell responses, but CD8⁺ T cells isolated from OVA_{PEP}-SLNP@CpG immunized mice of the present invention produced much higher levels of INF-γ and granzyme B (see FIGS. 25-26).

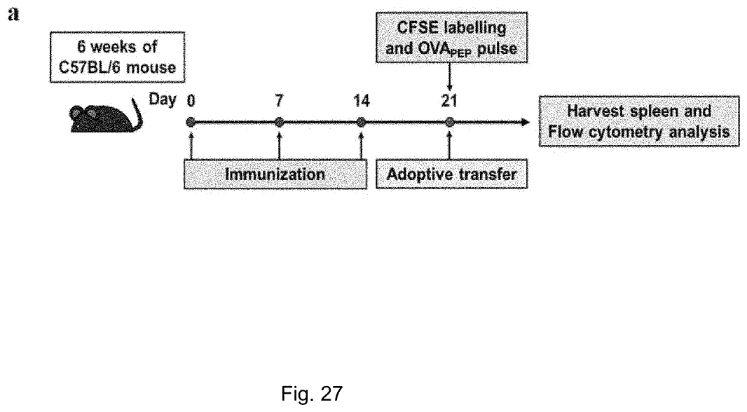

The in vivo antigen-specific killing activity of these CD8⁺ T cells was assessed by adoptively transferring of a mixture of half splenocytes obtained from nonpulsed mice and half splenocytes pulsed with OVAPEP to recipient mice immunized with the respective vaccine regimens. After 18 hours of adoptive transfer, the antigen-specific killing ability of CD8⁺ T cells was assessed by flow cytometry (see FIG. 27).

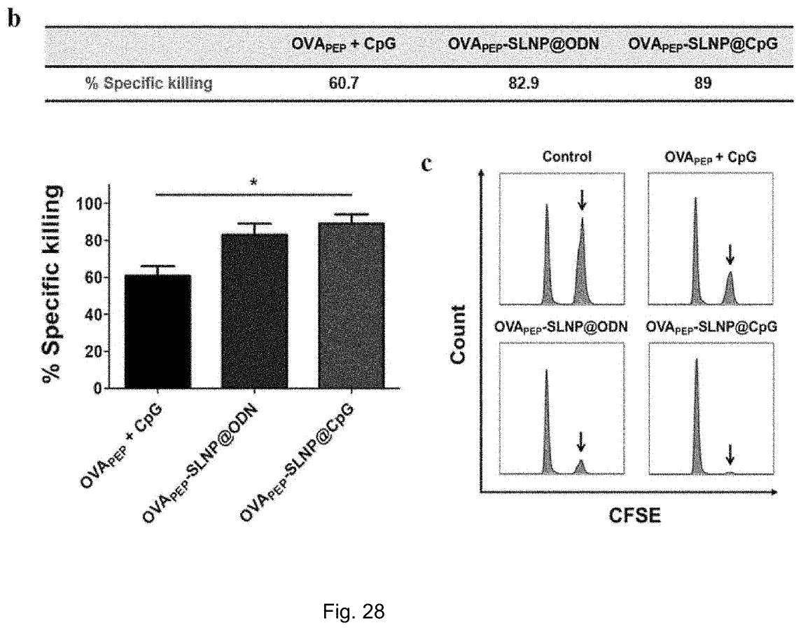

Percentage of OVA_{PEP}-specific killing of metastasized splenocytes in mice immunized with OVA_{PEP}-SLNP©CpG (89%) than in mice immunized with soluble OVA_{PEP}+CpG (60.7%) or OVA_{PEP}-SLNP@ODN (82.9%). was higher (see FIG. 28).

Taken together, these results suggest that OVA_{PEP}-SLNP@CpG can induce much higher antigen-specific killing activity in CD8⁺ T cells than physical mixture of soluble antigen+adjuvant,

### Example 5: Prophylactic Effect: Tumor Prevention by OVA_{PEP}-SLNP@CpG Nanovaccine

The present inventors investigated the in vivo effect of each vaccine regimen to prevent tumor growth using two mouse lymphoma cell lines, EL4 and E.G7-OVA. E.G7-OVA was derived from EL4 cells by transfection of the OVA gene.

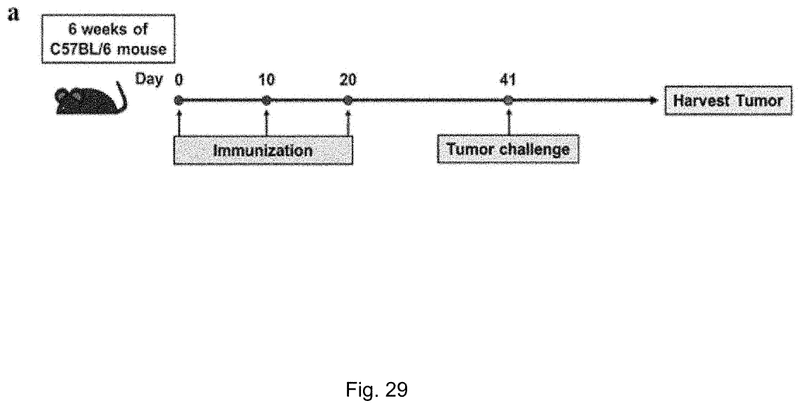

First, mice were immunized three times at 10-day intervals with each of the four vaccine modalities. Three weeks after the third immunization, EL4 and E.G7-OVA cells were injected into the contralateral flanks of these mice, respectively (see FIG. 29).

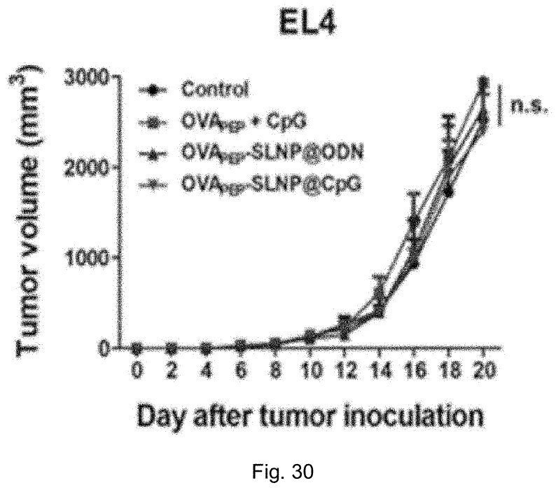

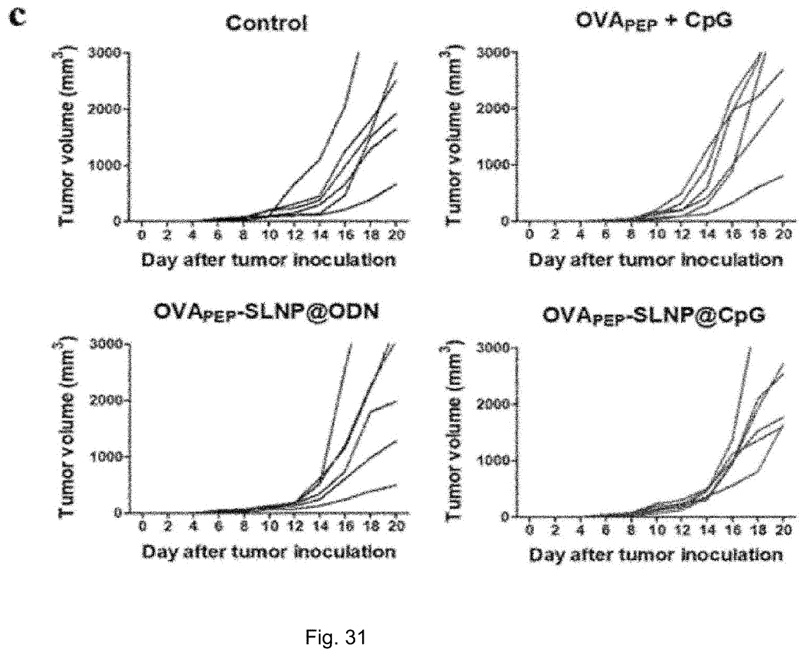

Although OVA_{PEP}-SLNP@CpG did not inhibit the growth of EL4-derived tumors, the growth of E.G7-OVA-derived tumors in the contralateral flank was completely prevented (FIGS. 30-31).

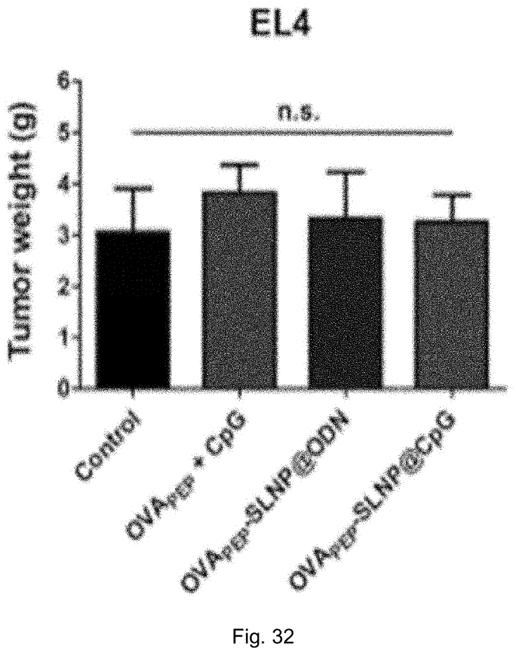

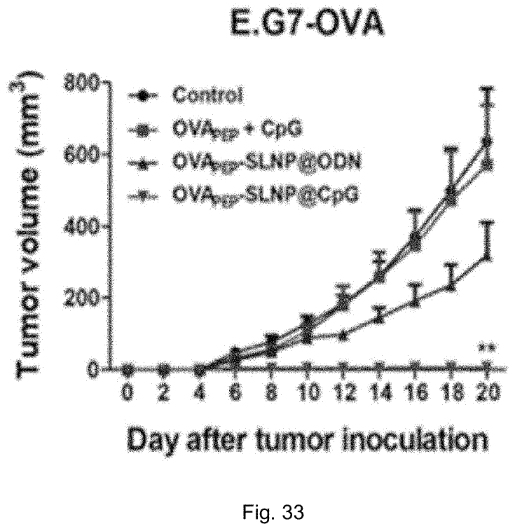

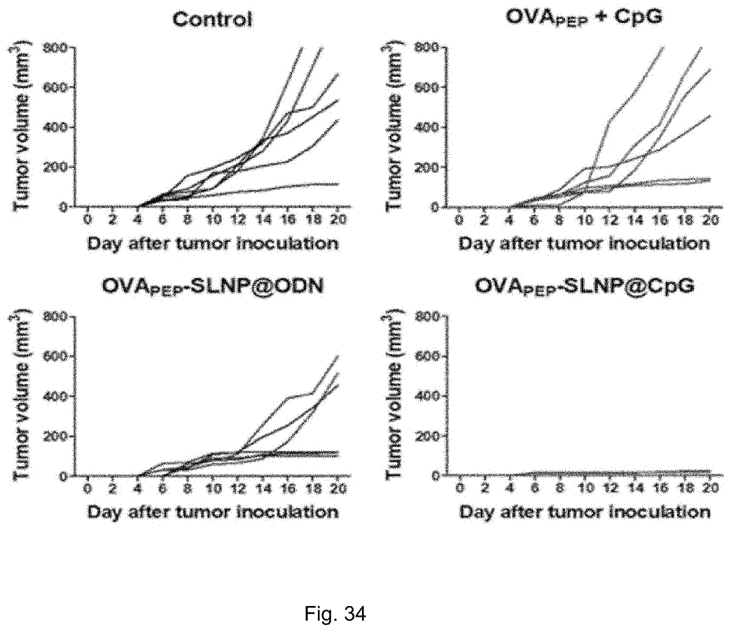

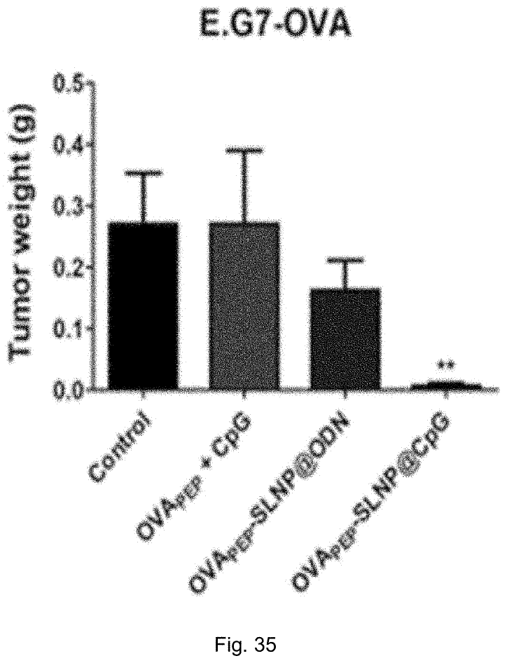

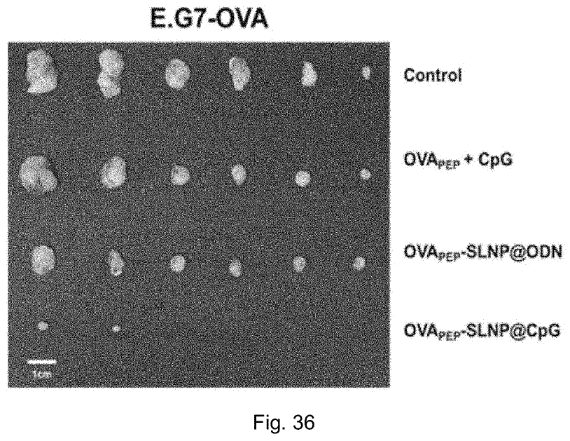

Soluble OVA_{PEP}+CpG had no effect in preventing tumor growth in the two cell lines, but OVA_{PEP}-SLNP@ODN was moderately effective against both, but not more effective than OVA_{PEP}-SLNP@CpG against E.G7-OVA-derived tumors (FIGS. 32-36).

This finding means that the nanovaccine of the present invention prevented tumor growth in an antigen-specific manner.

### Example 6: Therapeutic Efficacy of OVA_{PEP}-SLNP@CpG in an Established Tumor Model

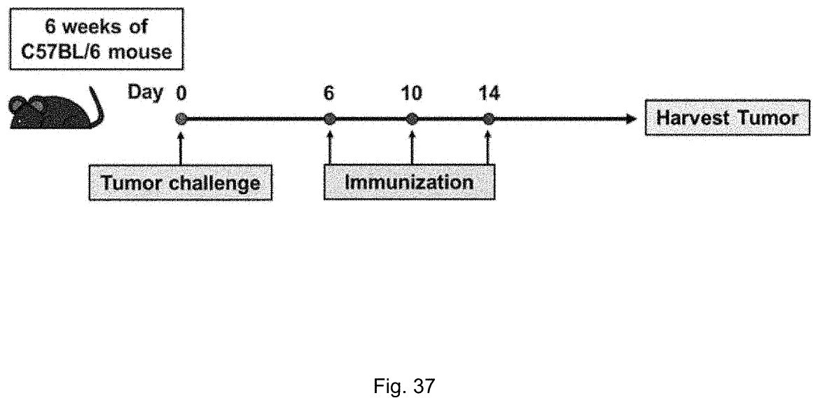

Next, the present inventors evaluated the therapeutic efficacy of the OVA_{PEP}-SLNP@CpG nanovaccine of the present invention in E.G7-OVA tumor-bearing mice. When the average tumor volume reached ˜50 mm³, mice were randomly divided into 4 groups and immunized 3 times at 4-day intervals with each vaccine modality (see FIG. 37).

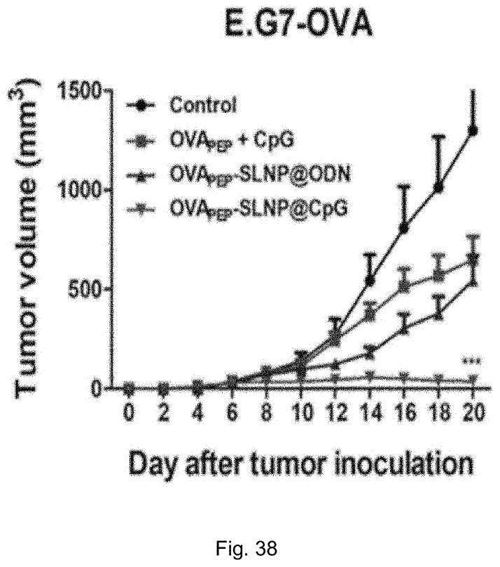

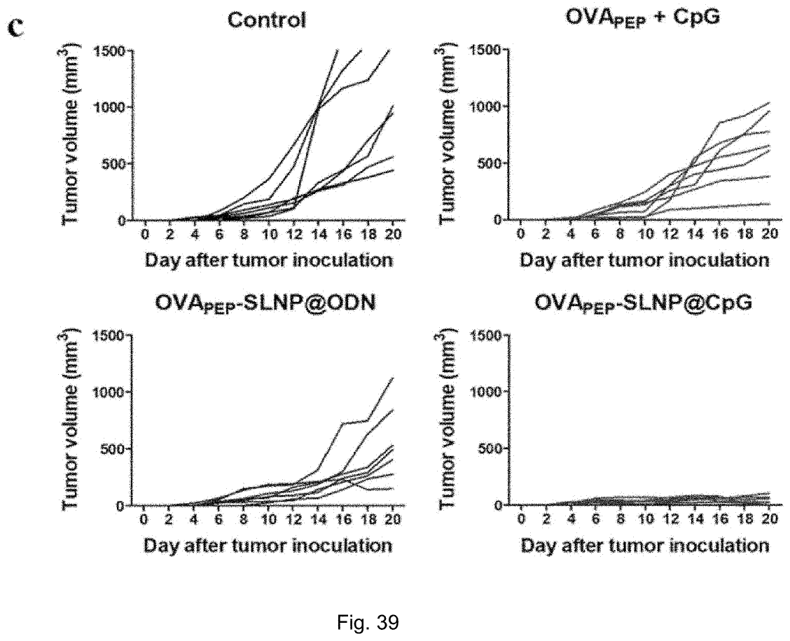

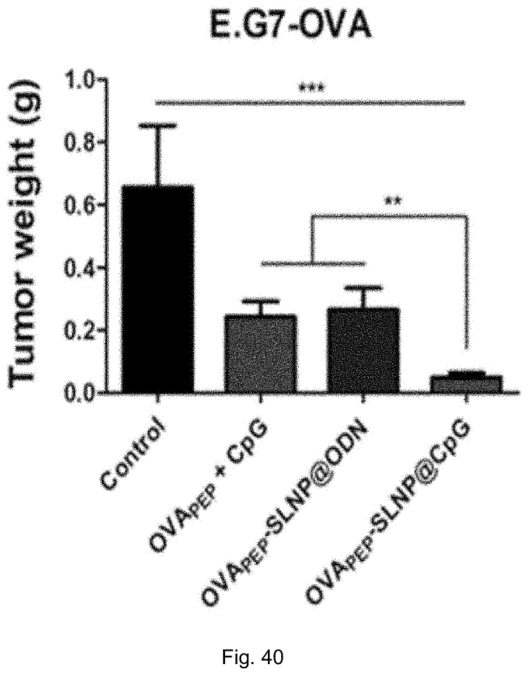

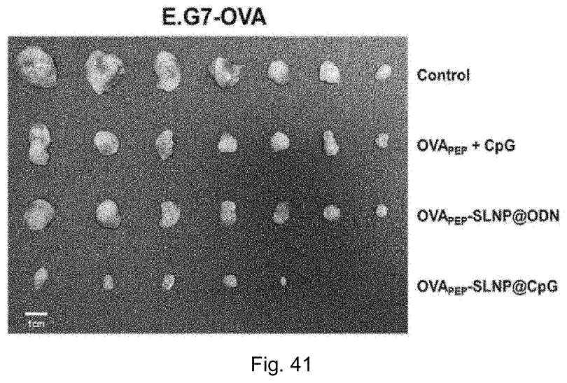

Immunization induced with soluble OVA_{PEP}+CpG or OVA_{PEP}-SLNP@DN showed only moderate tumor growth inhibition compared to vehicle control, but the immunization induced with OVA_{PEP}-SLNP@CpG of the present invention significantly inhibited tumor growth, and two of the seven mice lacked a tumor (see FIGS. 38-41).

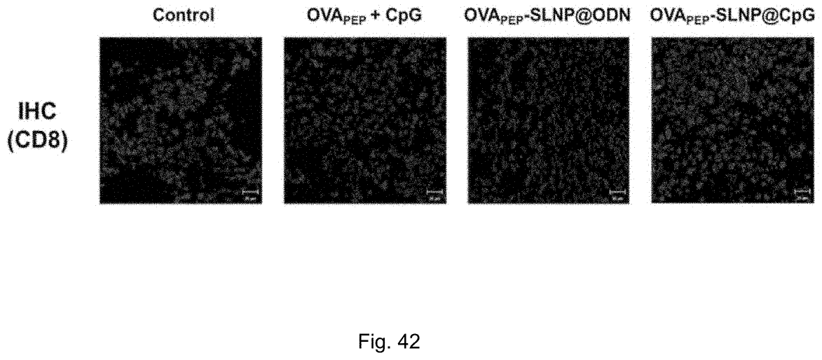

Histopathological analysis of tumor tissues was performed to better understand the effects of nanovaccines at the cellular level, Immunohistochemistry (IHC) showed that CD8⁺ T cell infiltration was much higher in tumor tissues from mice immunized with our OVA_{PEP}-SLNP@CpG of the present invention than in other groups (see FIG. 42).

Although the tumor tissue was analyzed during the late rejection phase of the CTL response, the difference in the degree of T cell infiltration between groups was significant.

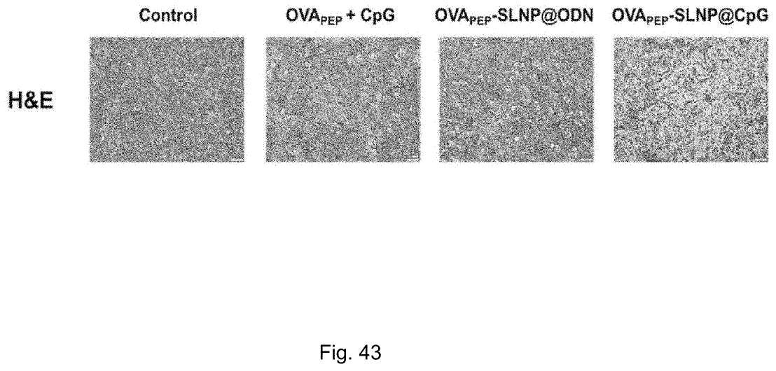

Hematoxylin and eosin (H&E) staining of tumor tissues showed that massive cell damage such as altered nuclei, enucleated necrotic cells and dead cell-derived debris occurred in the OVA_{PEP}-SLNP@CpG-immunized group of the present invention, but this was not the case in the other groups (see FIG. 43).

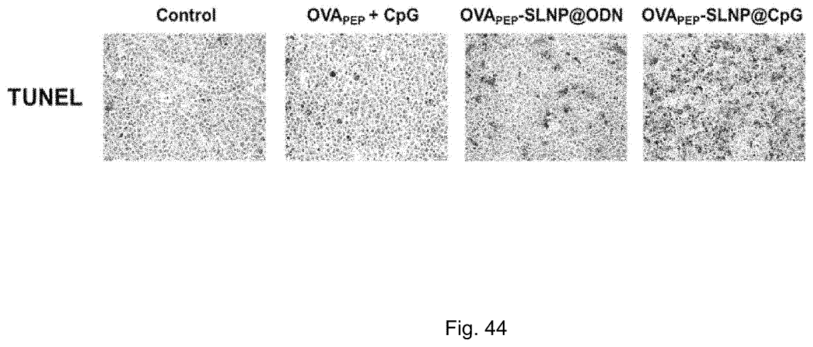

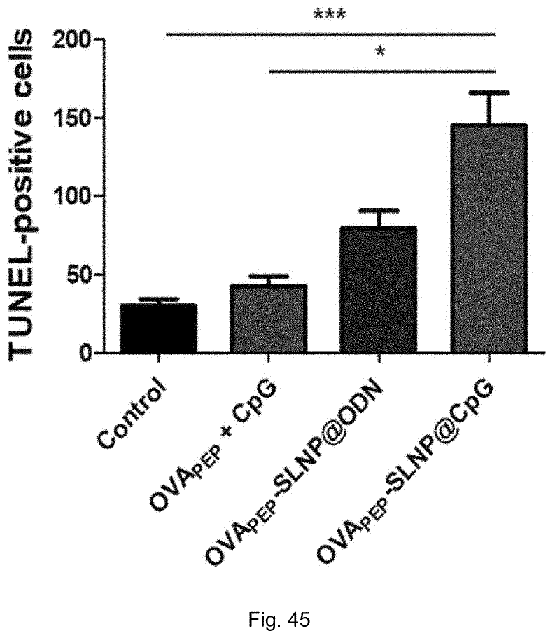

Terminal deoxynucleotidyl-transferase-mediated dUTP nick-end labeling (TUNEL) assay confirmed massive apoptosis in tumor tissues from the OVA_{PEP}-SLNP@CpG-immunized group of the present invention (see FIGS. 44-45).

Taken together, these histopathological analysis indicate that the therapeutic efficacy of OVA_{PEP}-SLNP@CpG immunization is caused by the mass death of cancer cells according to increased T cell infiltration into the tumor.

### Example 7: Tumor Regrowth Inhibitory Effect of Sequential Combination of Immune Checkpoint Blocking and OVA PEP-SLNP tx. CpG Nanovaccine

Although the OVA_{PEP}-SLNP@ CpG nanovaccine was highly effective in preventing and inhibiting tumor growth, the therapeutic response varied in individual mice. To evaluate the change in antitumor effect, tumors obtained from OVA_{PEP}-SLNP@CpG-immunized mice were arbitrarily divided into two groups based on their relative size: large tumor group (>˜60 mm³, two of seven mice) and small tumor group (<˜60 mm³, three of seven mice).

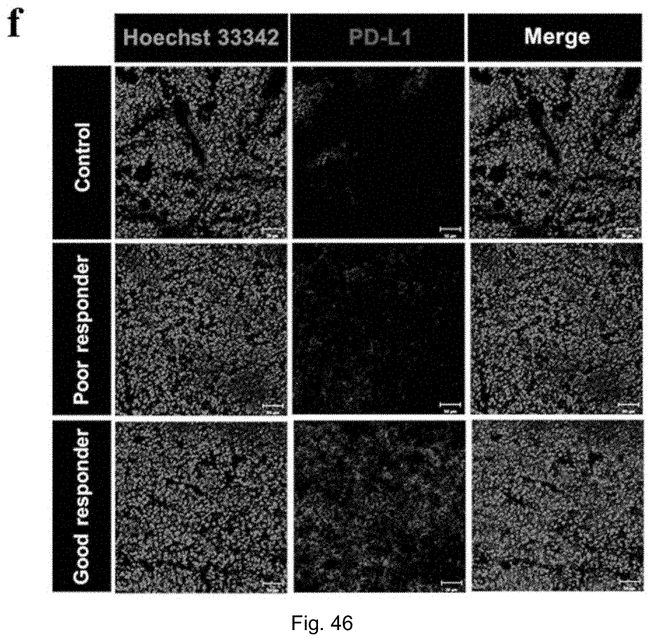

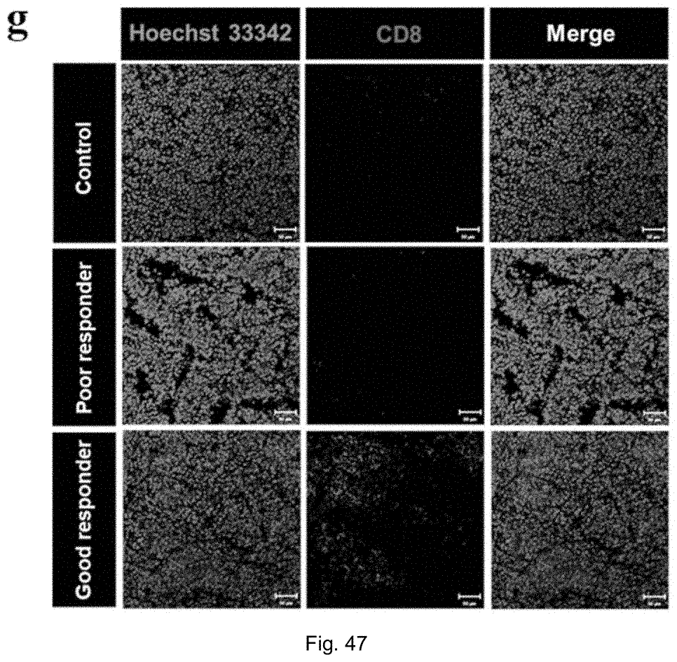

IHC showed that PD-L1 expression was significantly higher in small tumor groups (classified as ‘good responders’) than in large tumor groups (classified as ‘poor responders’) or unvaccinated controls (see FIG. 46). Additionally, CD8⁺ T cell infiltration was much better in good responders than in poor responders or unvaccinated mice (see FIG. 47),

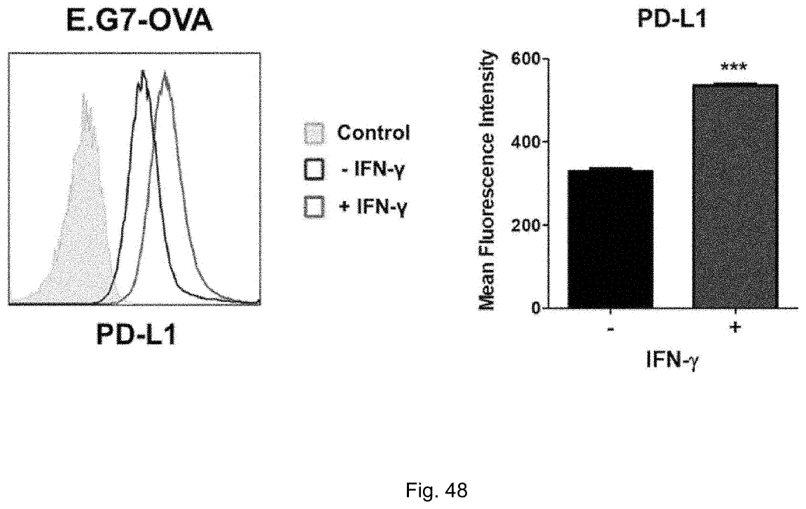

The present inventors have also found that PD-L1 expression in E.G7-OVA cancer cells is markedly induced by treatment with IFN-γ, which is a typical antitumor cytokine secreted by activated CD8+ T cells (FIG. 48).

Since PD-L1 expression in TME is increased by IFN-γ secreted from T cells, these findings suggest that greater antitumor efficacy in good responders may result from higher CD8⁺ T cell infiltration and cancer cell death by IFN-γ. However, since PD-L1 expression and co-localization of tumor-infiltrating T cells in tumor tissues are closely related to adaptive immune suppression and resistance, good responders with high PD-L1 induction in tumors can develop adaptive immune resistance through T cell depletion, leading to tumor recurrence. These findings suggest that new combinatorial strategies involving the order and timing of treatment of cancer nanovaccines and ICBs, need to be investigated.

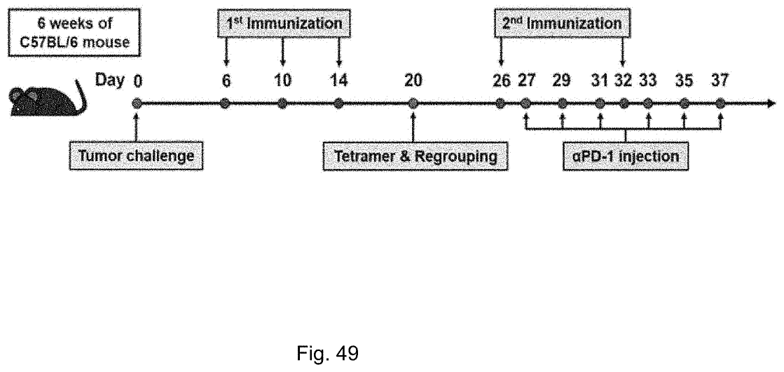

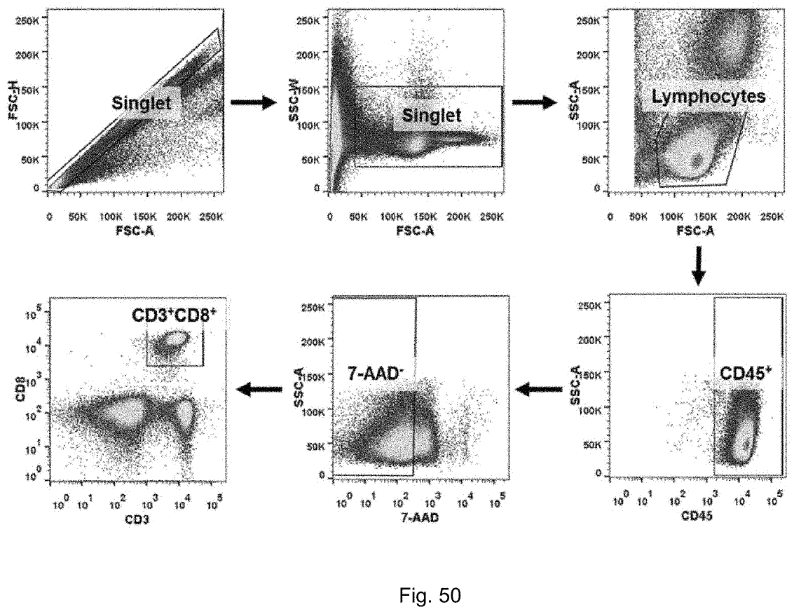

To investigate the validity of sequentially combining nanovaccine with ICB therapy, 50 mice were vaccinated twice with or without antibody to mouse PD-1 (αPD-1), Specifically, after inoculation to the side with E.G7-OVA cancer cells, OVA_{PEP}-SLNP@CpG nano-vaccine was subcutaneously injected 3 times after 6 days to perform a first immunization (FIG. 49). Twenty days after inoculation with E.G7-OVA cancer cells, mice were divided into two groups based on the therapeutic response to the nanovaccine. 10 mice (20%) were poor respondersm and 40 mice (80%) were good responders. CD8⁺ T cells were isolated from both poor and good responders, and their phenotype was analyzed by flow cytometry using a gating strategy (see FIG. 50).

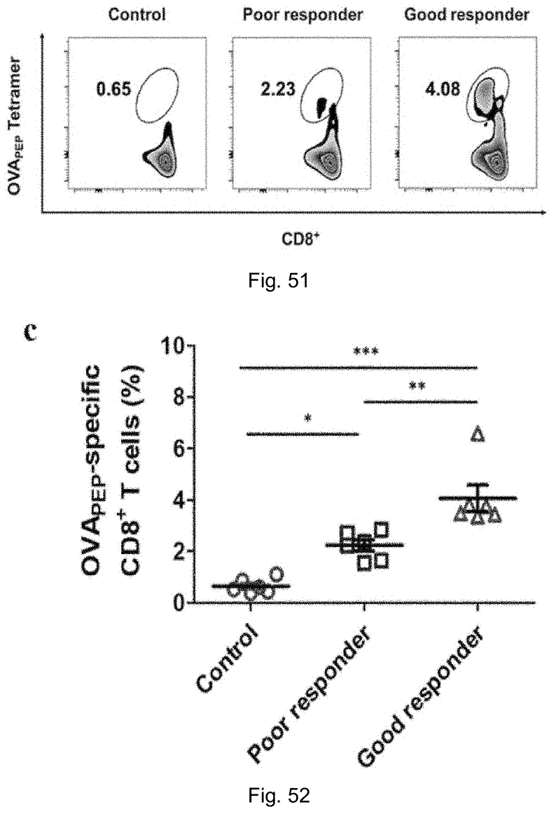

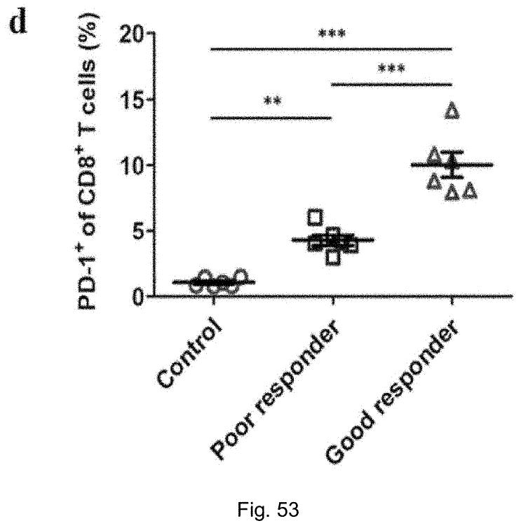

Tetramer assay showed that the percentage of OVA_{PEP} (SIINFEKL)-specific CD8⁺ T cells was approximately 2-fold higher in good responders than in poor responders and unimmunized controls (see FIGS. 51-52). Additionally, expression of PD-1 by CD8⁺ T cells was much higher in good responders than in poor responders and unvaccinated controls (see FIG. 53).

This finding is in good agreement with reports showing that PD-1 expression is upregulated in antigen-specific CD8+ T cells induced by vaccination,

It is in good agreement with reports showing that it can be considered to reflect T cell depletion and activation, which can be considered to reflect T cell depletion and activation. The number of PD-1⁺ CD8+ T cells in TME was shown to be positively correlated with the number of these cells in peripheral blood. Further, infiltration of PD-1⁺ CD8⁺ T cells into the tumor was reported to be a positive marker for response to ICB therapy. The number of PD-1⁺ CD8⁺ T cells in peripheral blood was high in good responders (see FIG. 53), and PD-L1 was expressed in tumor tissue (FIG. 46), so that it seemed reasonable to treat only good responders with αPD-1.

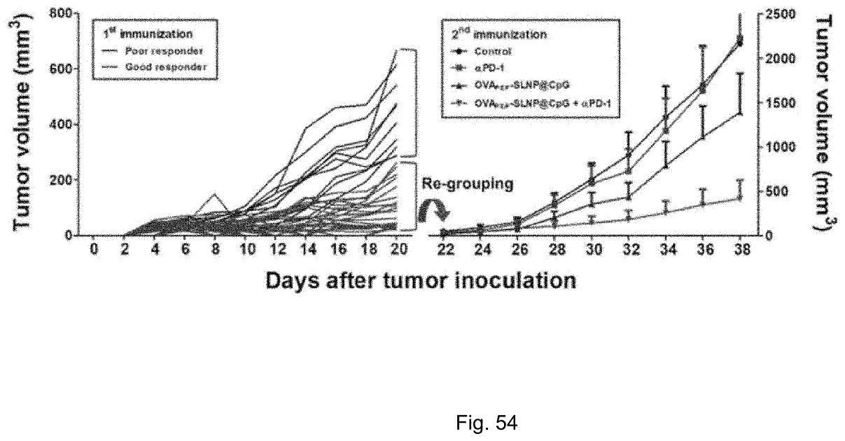

The 40 good responders were randomly divided into 4 groups of 10 mice each (see FIGS. 49 and 54).

i) one group was vehicle control, ii) another was treated with αPD-1 alone and iii) the other was reimmunized with OVA_{PEP}-SLNP@CpG, and iv) the other was reimmunized with OVA_{PEP}-SLNP@CpG and treated with αPD-1.

Starting on day 26, the last two groups of mice were immunized twice with the OVA_{PEP}-SLNP@CpG of the invention at 6-day intervals, and the second and fourth groups of mice received 6 intraperitoneal injections of αPD-1 at 2-day intervals.

Only the vehicle control showed rapid regrowth of the tumor within a few days (see FIG. 54), which is presumed to be because the tumor recurrence cannot be controlled as a result of the depletion of antigen-specific T cells in the tumor.

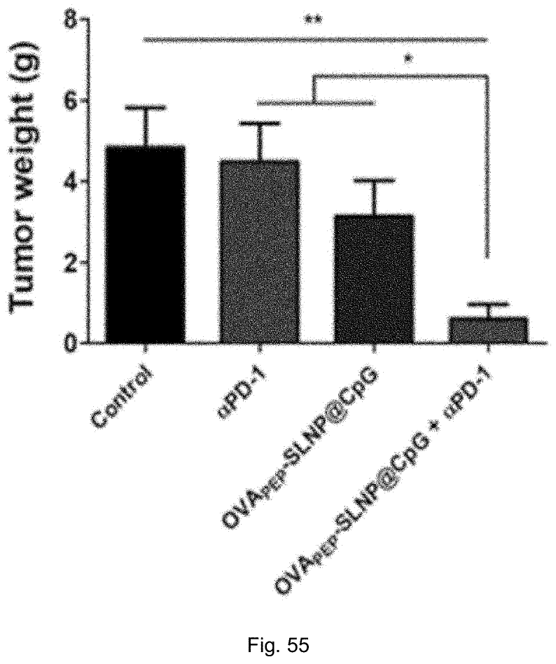

Unlike the initial expectations by the present inventors, αPD-1 alone, which was expected to revitalize exhausted PD-1⁺ CD8⁺ T cells, could hardly inhibit tumor regrowth. Because antigen-specific T cells have only short-term activity, an additional nanovaccine was needed to boost the CTL response again. The second cycle of OVA_{PEP}-SLNP@CpG alone immunization of the present invention partially inhibited tumor regrowth, but its efficacy was not significantly different compared to the vehicle control group or the αPD-1 group, which suggests that when PD-L1 expression is induced to high levels in tumors after the first immunization cycle, the treatment results of the second cycle immunization with the nanovaccine appear to be poor. In contrast, tumor regrowth was effectively inhibited by the combination of the OVA_{PEP}-SLNP@CpG nanovaccine of the present invention+the second cycle of αPD-1 (see FIGS. 54-55).

These results suggest that the effect of combination therapy differs depending on the administration order and timing of nanovaccine and ICB therapy.

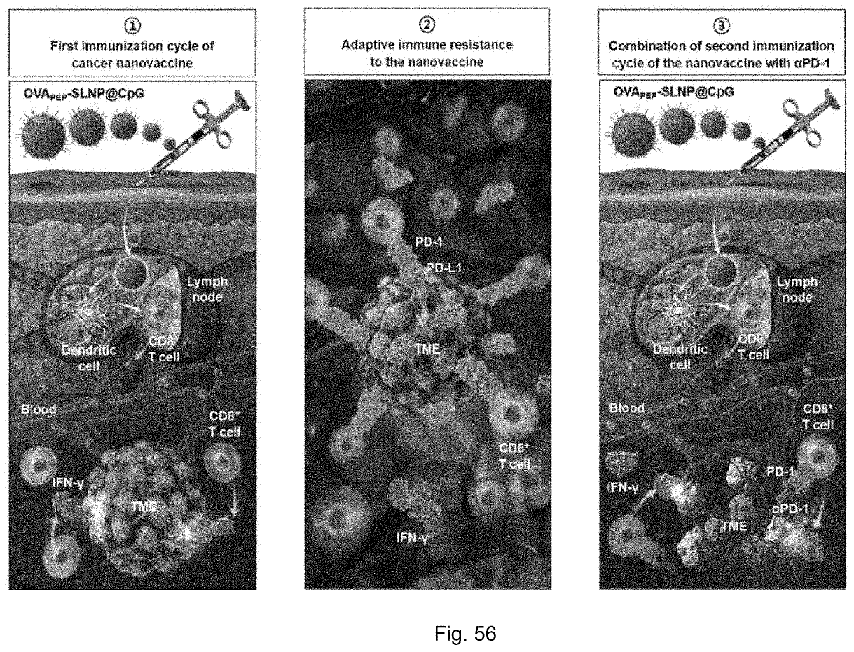

That is, initial immunization with nanovaccines can result in high tumor growth inhibition, but at the same time, it can induce tumor expression of PD-L1 and lead to antigen-specific T cell depletion. When treating a good responder for first-cycle immunization with a combination of second-cycle nanovaccine immunization+ICB it can lead to a strong therapeutic response (see FIG. 56).

### Discussion

Cancer nanovaccines using nanomaterials as antigen and/or adjuvant-delivery carriers can induce tumor antigen-specific T cell immunity, and have shown potential as a therapeutic method in in vivo animal models. Additionally, the combination of ICB immunotherapy and cancer nanovaccine can further enhance the anti-tumor efficacy of cancer nanovaccine.

The lack of an optimal vaccination regimen that can address not only the problems related to antigen-delivering nanomaterials themselves, such as toxicity and manufacturability, but also the adaptive resistance of tumors to cancer vaccines has hampered the clinical application of cancer nanovaccines.

To solve these problems, the present inventors have developed novel antigen/adjuvant-delivery nanoparticles made of biocompatible lipid components. These nanoparticles, in combination with ICB immunotherapy, showed very strong antitumor efficacy in both prophylactic and therapeutic tumor models, and have demonstrated the validity of a new treatment regimen based on the order and timing of modalities that effectively suppress tumor recurrence.

The lack of toxicity associated with antigen-delivery nanomaterials and the antitumor efficacy of nanovaccines are key factors for successful clinical application. The present invention has been disclosed the construction of a cancer nanovaccine using a biocompatible and non-toxic naturally occurring or synthetic components. Two biocompatible and non-toxic neutrally charged phospholipids, i.e., DOPE and DSPE-PEG1000, have been widely used in clinically available liposome-based therapies. The present inventors showed in previous studies that MA-Chol, which is a cationic cholesterol derivative, can form stable complexes with oligonucleotides such as siRNA and CpG ODN. MA-Chol is biodegradable and non-toxic because it is synthesized from endogenous arginine and cholesterol via an ester bond. Actually, all three biocompatible and non-toxic components were able to successfully form SLNPs with CpG ODN, and the antigen/adjuvant-carrying nanovaccine (OVA_{PEP}-SLNP@CpG) was sufficiently stable in physiological media that it was released directly into the local LN according to local injection. These results shows that SLNP is clinically suitable for use in cancer nanovaccines. Further, the model tumor antigen (OVA_{PEP}) was linked to the SLNP surface via a disulfide bond, so that the intact antigen was released in the cytoplasm and effectively displayed on the MHC of APC. In fact, the OVA^{PEP}-SLNP@CpG nanovaccine presented antigens that were efficiently uptaken by DCs in vitro and in vivo and released on the DC surface via MHC, which made it possible to effectively cross-prime CD8⁺ T cells to antigen. Although the experimental conditions used in this example may be different from those of other nanovaccine systems, the antitumor efficacy of OVA_{PEP}-SLNP@CpG was impressive because 4 of 6 mice in the prophylactic tumor model and 2 of 7 mice in the therapeutic tumor model had no tumor. The efficacy of these nanovaccines may be due to their ability to induce strong CTL responses against antigen-expressing E.G7 tumors. OVA_{PEP}-SLNP©CpG is made of a biocompatible, non-toxic material and exhibits strong antitumor activity, so it has clinical potential for use as a therapeutic cancer nanovaccine.

The generation of adaptive resistance of tumors to cancer vaccines has interrupted clinical applications of cancer vaccines. PD-L1 expression and tumor-infiltrating CD8⁺ T cell colocalization are closely associated with adaptive immune resistance, This study focused on differences in therapeutic response to nanovaccines in individual E.G7 tumor-bearing mice. Tumor expression of PD-L1, infiltration of CD8⁺ T cells, and the ratio of circulating OVAPEP-specific/PD-1+CD8⁺ cells in peripheral blood were significantly higher in good responders than in poor responders and unvaccinated controls. This suggests that good responders can be regarded as ‘hot tumors’ that respond better to ICB therapy than ‘cold tumors’. On the other hand, these findings also indicated that good responders develop adaptive resistance to nanovaccines, leading to tumor recurrence if not properly treated. Therefore, only good responders were treated with the combination of nanovaccine and ICB therapy, Efforts were made to maximize treatment outcomes by varying the order and timing of each modality. The first cycle of vaccination was performed to systemically increase antigen-specific T cell immunity and induce PD-L1 expression in tumors. Good responders sensitive to ICB therapy were then treated with a second vaccination in combination with ICB antibody. However, treating good responders with αPD-1 alone was completely ineffective in inhibiting tumor regrowth, which suggests that booster vaccination is necessary to reactivate antigen-specific memory T cells derived from the first immunization cycle. Further, treating good responders with only the second cycle of OVA_{PEP}-SLNP@CpG vaccination was ineffective. This indicates that T cell depletion by high PD-L1 induction in tumors is possible. The present inventors have found that only the combination of αPD-1 with the second cycle of OVA_{PEP}-SLNP@CpG vaccination significantly improved the treatment outcome, amd resulted in effective inhibition of tumor regrowth or recurrence. This may be due to the re-boosting of antigen-specific T cell responses by the second vaccination, along with reversal of immune suppression by the ICB antibody. Taken together, these findings clearly indicate the importance of the treatment order and timing of each modality in combination therapies involving nanovaccines and ICB antibodies,

Despite their high therapeutic potential, cancer vaccines can stimulate cancer cells to produce immunosuppressive molecules and recruit immune regulatory cells to TME. For example, vaccine-induced CD8⁺ T cells upregulate PD-L1 and indoleamine-2,3-dioxygenase (IDO) expression and recruit T cells (Tregs) in a model of metastatic melanoma, thereby inducing immunosuppression. Further, cancer vaccines have been shown to upregulate the expression of NKG2A inhibitory receptors on tumor-infiltrating CD8⁺ T cells. Despite the presence of various inhibitory and immunosuppressive molecules, this study investigated the effect of nanovaccines on the expression of only one inhibitory molecule, PD-L1. Therefore, there is a need to investigate other immunosuppressive molecules induced by nanovaccines and their mechanisms of action. Furthermore, the reasons for the differences in therapeutic response to nanovaccines in individual mice with the same genetic background are unclear. Nevertheless, the results of the present invention indicate the importance of the order and timing of each modality in designing combination immunotherapy comprising nanovaccines. Although tumor size may not be a good marker for differentiating good and poor responders, the sequential combination strategy proposed in this study requires additional clinical evaluation of personalized therapy. Imaging modalities such as computer tomography to monitor tumor size and positron emission tomography to monitor tumor activity can be a criterion for distinguishing between good and bad responders.

In conclusion, the present inventors have developed a novel type of antigen/adjuvant-carrying nanovaccine composed of biocompatible and non-toxic lipid components. These nanovaccines showed very strong antitumor efficacy in both prophylactic and therapeutic tumor models. Further, a novel combination treatment regimen consisting of cancer nanovaccine and ICB immunotherapy was proposed according to the treatment order and timing. Such protocols can improve the persistence of anti-tumor immunity, including effective inhibition of tumor growth and recurrence. These findings further suggest the necessity for evaluating these new combination therapy regimens in other immunotherapy modalities.

1. A lipid nanoparticle comprising an antigen, a phospholipid, a cationic lipid, and an adjuvant.

2. The lipid nanoparticle of claim 1, wherein the antigen is a tumor-associated antigen.

3. The lipid nanoparticle of claim 1, wherein the phospholipid is a phospholipid having 14 to 22 aliphatic carbon atoms.

4. The lipid nanoparticle of claim 1, wherein the cationic lipid is at least one cationic lipid selected from the group consisting of Dimethyldioctadecyl-ammoniumbromide (DDAB), dimethyldioctadecylammonium (DDAB), (N,N-dimethyl-N-([2-sperminecarboxamido]ethyl)-2,3-bis(dioleyloxy)-1-propaniminium pentahydrochloride) (DOSPA), (N-[1-(2,3-dioleyloxy)propyl]-N, N, N-trimethylammonium) (DOTMA), (N-[1-(2,3-dioleoyloxy)propyl]-N,N,N-trimethylammonium) (DOTAP), 3β-[N-(N′,N′-dimethylaminoethane)-carbamoyl]cholesterol (DC-Chol), N4-cholesteryl-Spermine (GL67), 1,2-dioleyloxy-3-dimethylaminopropane (DODMA), O-alkyl phosphatidylcholines derivative, and dimethylammonium-propane (DAP) derivative.

5. The lipid nanoparticle of claim 1, wherein the cationic lipid is a cationic cholesterol derivative.

6. The lipid nanoparticle of claim 5, wherein the cationic cholesterol derivative is monoarginine-cholesterol (MA-Chol).

7. The lipid nanoparticle of claim 1, wherein the adjuvant is immunostimulatory single- or double-stranded oligonucleotide, immunostimulatory small-molecule compound, or a combination thereof.

8. The lipid nanoparticle of claim 7, wherein the single- or double-stranded oligonucleotide is a CpG oligonucleotide, a STING -active oligonucleotide, or a combination thereof.

9. A vaccine composition comprising the lipid nanoparticle claim 1 as an active ingredient.

10. The vaccine composition of claim 9, wherein the vaccine composition is for preventing or treating cancer.

11. A cancer vaccine kit which comprises a lipid nanoparticle including a tumor-associated antigen, a phospholipid, a cationic lipid, and an anionic drug as a first vaccine composition; and
comprises the lipid nanoparticle and an immune checkpoint inhibitor as a second vaccine composition.

12. The cancer vaccine kit of cairn 11, wherein the immune checkpoint inhibitor is an anti-PD-1 antibody or an anti-PD-L1 antibody.
